(19) United States
(12) Patent Application Publication
(10) Pub. No.: US 2024/0074686 A1
(43) Pub. Date: Mar. 7, 2024

(54) METHOD OF OBTAINING URINATION INFORMATION AND DEVICE THEREOF

(71) Applicant: DAIN TECHNOLOGY, INC., Seoul (KR)

(72) Inventors: Jee Young SONG, Seoul (KR); Kyeong Yeon DOO, Seoul (KR); Ji Young JUNG, Gyeonggi-do (KR); Daeyeon KIM, Seoul (KR)

(21) Appl. No.: 18/486,510

(22) Filed: Oct. 13, 2023

Related U.S. Application Data
(63) Continuation of application No. PCT/KR2023/001428, filed on Jan. 31, 2023.

(30) Foreign Application Priority Data
Sep. 2, 2022 (KR) 10-2022-0111723

(51) Int. Cl. A61B 5/20

(52) U.S. Cl. CPC A61B 5/208

(57) ABSTRACT

A method of obtaining urination information and a device thereof are proposed. The method includes obtaining one or more first feature data by using first sound data, obtaining a urine volume determination value by using the one or more first feature data and a pre-trained urine volume determination model, obtaining a urine flow rate determination value by using the one or more first feature data and a pre-trained urine flow rate determination model, and obtaining urine flow rate information by reflecting a ratio of an estimated urine volume calculated based on the urine flow rate determination value and the urine volume determination value to the urine flow rate determination value.

### CROSS-REFERENCE TO RELATED APPLICATIONS

This application is a Continuation of PCT International Application No. PCT/KR2023/001428, filed Jan. 31, 2023, which claims priority to Korean Patent Application No. 10-2022-0111723, filed Sep. 2, 2022, the entire contents of which are hereby expressly incorporated by reference.

### TECHNICAL FIELD

The present specification relates to a method of obtaining urination information and, more particularly, to a method of extracting urination information from urination sound by using a urine volume determination model, a urination presence/absence determination model, and a urine flow rate determination model.

### BACKGROUND ART

Urination information, such as urine flow rates and urine volumes, is required to check the health conditions of an individual's urinary system organs.

However, since there were no convenient methods of obtaining urination information previously, the urination information was obtained through separate physical measurement methods, such as using a urine collection cup or a toilet capable of measuring weight. Accordingly, environments where the urination information is obtainable were limited, and also measurement methods provided therein were inconvenient and unsustainable, so it was difficult to continually monitor an individual's conditions.

Accordingly, in order to continually monitor an individual's health, it was essential to develop a method of obtaining accurate urination information conveniently.

Under the support of the Ministry of Science and ICT, the Ministry of Trade, Industry and Energy, the Ministry of Health and Welfare, and the Ministry of Food and Drug Safety, the present technology is developed with (Fdn.) Interdepartmental Full-Cycle Medical Device R&D project Group as the project management specialized organization, and is a technology developed through the development of Artificial Intelligence-based Digital Therapeutics for Voiding Dysfunction in “Interdepartmental Full-Cycle Medical Device R&D project “(Project number: RS-2020-KD000141, Project identification number: 9991006814). The project implementation agency is Dyn Technology Co., Ltd., and the research period is from Sep. 1, 2020 to Dec. 31, 2023.

### DISCLOSURE

### Technical Problem

An objective of the present disclosure for solving the problem is to provide a method and device of obtaining highly accurate urination information from sound data obtained by recording sound during a person's urination process.

Another objective of the present disclosure for solving the problem is to provide a method and device of obtaining highly accurate urination information by estimating a urine volume from sound data.

The problem to be solved in the present disclosure is not limited to the above-mentioned problem, and the problems not mentioned will be clearly understood by those skilled in the art to which the present disclosure belongs from the present specification and accompanying drawings.

### Technical Solution

According to one embodiment of the present disclosure, a method of obtaining urination information may be provided, wherein the method of obtaining urination information includes obtaining one or more first feature data by using first sound data, wherein the first sound data reflect a sound of a urination process; obtaining a urine volume determination value by using the one or more first feature data and a pre-trained urine volume determination model, wherein the urine volume determination model is trained with a urine volume training data set, wherein the urine volume training data set comprises one or more second feature data generated based on second sound data recorded during a urination process and a value related to a urine volume corresponding to the second sound data; obtaining a urine flow rate determination value by using the one or more first feature data and a pre-trained urine flow rate determination model, wherein the urine flow rate determination model is trained with a urine flow rate training data set, wherein the urine flow rate training data set comprises one or more third feature data generated based on third sound data recorded during a urination process and a value related to a urine flow rate corresponding to the third sound data; and obtaining a urine flow rate information by reflecting a ratio of an estimated urine volume calculated based on the urine flow rate determination value and the urine volume determination value to the urine flow rate determination value.

According to another embodiment of the present disclosure, a method of obtaining urination information may be provided, wherein the method of obtaining urination information includes obtaining one or more first feature data and one or more first relative feature data by using first sound data, wherein the first sound data reflect a sound of a urination process, and the first relative feature data comprise normalized value; obtaining a urine volume determination value by using the one or more first feature data and a pre-trained urine volume determination model, wherein the urine volume determination model is trained with a urine volume training data set, wherein the urine volume training data set comprises one or more second feature data generated based on second sound data recorded during a urination process and a value related to a urine volume corresponding to the second sound data; obtaining a relative urine flow rate determination value by using the one or more first relative feature data and pre-trained relative urine flow rate determination model, wherein the relative urine flow rate determination model is trained with a relative urine flow rate training data set, wherein the relative urine flow rate training data set comprises one or more second relative feature data generated based on third sound data recorded during a urination process and a value related to a relative urine flow rate corresponding to the third sound data; and obtaining a urine flow rate information by reflecting a ratio of an integral value calculated based on the relative urine flow rate determination value and the urine volume determination value to the relative urine flow rate determination value.

According to another embodiment of the present disclosure, a sound analysis system may be provided, wherein the sound analysis system includes a memory storing first sound data, pre-trained urine volume determination model and pre-trained urine flow rate determination model, wherein the first sound data reflect a sound of a urination process, the urine volume determination model is trained with a urine volume training data set comprising one or more first feature data generated based on second sound data recorded during a urination process and a value related to a urine volume corresponding to the second sound data, and the urine flow rate determination model is trained with a urine flow rate training data set comprising one or more second feature data generated based on third sound data recorded during a urination process and a value related to urine flow rate corresponding to the third sound data; and at least one processor, wherein the processor obtain one or more third feature data by using the first sound data, obtain a urine volume determination value by using the one or more third feature data and the urine volume determination model, obtain a urine flow rate determination value by using the one or more third feature data and the urine flow rate determination model, and obtain a urine flow rate information by reflecting a ratio of an estimated urine volume calculated based on the urine flow rate determination value and the urine volume determination value to the urine flow rate determination value.

According to another embodiment of the present disclosure, a sound analysis system may be provided, wherein the sound analysis system includes a memory storing first sound data, pre-trained urine volume determination model and pre-trained relative urine flow rate determination model, wherein the first sound data reflect a sound of a urination process, the urine volume determination model is trained with a urine volume training data set comprising one or more first feature data generated based on second sound data recorded during a urination process and a value related to a urine volume corresponding to the second sound data, and the relative urine flow rate determination model is trained with a relative urine flow rate training data set comprising one or more first relative feature data generated based on third sound data recorded during a urination process and a value related to relative urine flow rate corresponding to the third sound data; and at least one processor, wherein the processor obtain one or more second feature data and one or more second relative feature data by using the first sound data, obtain a urine volume determination value by using the one or more second feature data and the urine volume determination model, obtain a relative urine flow rate determination value by using the one or more second relative feature data and the relative urine flow rate determination model, and obtain a urine flow rate information by reflecting a ratio of an integral value calculated based on the relative urine flow rate determination value and the urine volume determination value to the relative urine flow rate determination value.

The problem solutions of the present disclosure are not limited to the above-described solutions, and solutions that are not mentioned may be clearly understood to those skilled in the art to which the present disclosure belongs from the present specification and the accompanying drawings.

### Advantageous Effects

According to an exemplary embodiment, the accuracy of urine flow rate prediction may be improved by adjusting a result of the urine flow rate prediction by using urine volume prediction values predicted by using sound data.

According to another exemplary embodiment, when urine flow rate prediction values are obtained by using sound data, the accuracy of urine flow rate prediction may be increased by using a model for predicting a relative urine flow rate.

The effects of the present disclosure are not limited to the above-described effects, and effects not mentioned herein may be clearly understood by those skilled in the art to which the present disclosure belongs from the present specification and accompanying drawings.

### DESCRIPTION OF DRAWINGS

FIG. 1 is a view illustrating a urination information obtaining system according to an exemplary embodiment.

FIG. 2 is a block diagram illustrating a configuration of the sound analysis system according to the exemplary embodiment.

FIG. 3 is a view illustrating a urine volume determination model according to the exemplary embodiment.

FIGS. 4 and 5 are views illustrating a training process of the urine volume determination model according to the exemplary embodiment.

FIG. 6 is a view illustrating a configuration of a urine volume determination module according to the exemplary embodiment.

FIG. 7 is a view illustrating sound data preprocessing according to the exemplary embodiment.

FIG. 8 is a view illustrating a urination presence/absence determination model according to the exemplary embodiment.

FIGS. 9 and 10 are views illustrating a training process of the urination presence/absence determination model according to the exemplary embodiment.

FIG. 11 is a view illustrating a configuration of a urination presence/absence determination module according to the exemplary embodiment.

FIG. 12 is a view illustrating a method of obtaining urine volume information according to the exemplary embodiment.

FIG. 13 is a flowchart illustrating the method of obtaining the urine volume information according to the exemplary embodiment.

FIG. 14 is a view illustrating an adjusted spectrogram according to the exemplary embodiment.

FIG. 15 is a view illustrating a urine flow rate determination model according to the exemplary embodiment.

FIGS. 16 and 17 are views illustrating a training process of the urine flow rate determination model according to the exemplary embodiment.

FIG. 18 is a view illustrating a configuration of a urine flow rate determination module according to the exemplary embodiment.

FIG. 19 is a view illustrating a method of obtaining urine flow rate information according to the exemplary embodiment.

FIG. 20 is a view illustrating applying urination presence/absence determination values according to the exemplary embodiment.

FIGS. 21, 22, 23, and 24 are views illustrating respective methods of obtaining urine flow rate information according to the exemplary embodiment.

FIG. 25 is a flowchart illustrating the method of obtaining the urine flow rate information according to the exemplary embodiment.

FIG. 26 is a view illustrating a relative urine flow rate determination model according to an exemplary embodiment.

FIGS. 27 and 28 are views illustrating a training process of the relative urine flow rate determination model according to the exemplary embodiment.

FIG. 29 is a view illustrating a configuration of a relative urine flow rate determination module according to the exemplary embodiment.

FIGS. 30 and 31 are views illustrating respective methods of obtaining urine flow rate information according to the exemplary embodiment.

FIG. 32 is a flowchart illustrating the method of obtaining the urine flow rate information according to the exemplary embodiment.

FIGS. 33, 34, 35, 36, 37, and 38 are views illustrating urination information obtained according to the exemplary embodiment.

### MODE FOR INVENTION

Exemplary embodiments described in the present specification are intended to clearly describe the idea of the present disclosure to those skilled in the art. Therefore, the present disclosure is not limited by the exemplary embodiments, and the scope of the present disclosure should be interpreted as encompassing modifications and variations without departing from the idea of the present disclosure.

Terms used in the present specification are selected from among general terms, which are currently widely used, in consideration of functions in the present disclosure and may have meanings varying depending on intentions of those skilled in the art, customs in the field of art, the emergence of new technologies, or the like. However, in contrast, in a case where a specific term is defined and used with an arbitrary meaning, the meaning of the term will be described separately. Accordingly, the terms used in the present specification should be interpreted on the basis of the actual meanings and the whole context throughout the present specification rather than based on just names for the terms.

Numbers (e.g., first, second, etc.) used in a process of describing the present specification are merely identification symbols for distinguishing one component from other components.

In addition, the words “module” and “part/unit” used as noun suffixes for the components used in the following exemplary embodiments are given or mixed in consideration of merely the ease of writing the specification, and do not have distinct meanings or roles by themselves.

In the exemplary embodiments below, the singular forms are intended to include the plural forms as well, unless the context clearly indicates otherwise.

In the following exemplary embodiments, terms such as “comprise”, “include”, or “have” mean that a feature or a component described in the specification is present, and the possibility that one or more other features or components may be added is not precluded.

The accompanying drawings of the present specification are intended to easily describe the present disclosure, and shapes shown in the drawings may be exaggerated as necessary in order to facilitate in understanding the present disclosure. Therefore, the present disclosure is not limited by the drawings.

Where certain exemplary embodiments are otherwise implementable, a specific process order may be performed different from the described order. For example, two processes described in succession may be performed substantially and simultaneously, or may be performed in an order opposite to the described order.

When it is determined that detailed descriptions of well-known components or functions related to the present disclosure may obscure the subject matter of the present disclosure, detailed descriptions thereof may also be omitted herein as necessary.

According to one embodiment of the present disclosure, a method of obtaining urination information may be provided, wherein the method of obtaining urination information includes obtaining one or more first feature data by using first sound data, wherein the first sound data reflect a sound of a urination process; obtaining a urine volume determination value by using the one or more first feature data and a pre-trained urine volume determination model, wherein the urine volume determination model is trained with a urine volume training data set, wherein the urine volume training data set comprises one or more second feature data generated based on second sound data recorded during a urination process and a value related to a urine volume corresponding to the second sound data; obtaining a urine flow rate determination value by using the one or more first feature data and a pre-trained urine flow rate determination model, wherein the urine flow rate determination model is trained with a urine flow rate training data set, wherein the urine flow rate training data set comprises one or more third feature data generated based on third sound data recorded during a urination process and a value related to a urine flow rate corresponding to the third sound data; and obtaining a urine flow rate information by reflecting a ratio of an estimated urine volume calculated based on the urine flow rate determination value and the urine volume determination value to the urine flow rate determination value.

The estimated urine volume may be calculated by integrating the urine flow rate determination value over time.

The method of obtaining urination information may include obtaining a urination presence/absence determination value by using the first feature data and a pre-trained urination presence/absence determination model, wherein the urination presence/absence determination model is trained with a urination presence/absence training data set, wherein the urination presence/absence training data set comprises one or more fourth feature data generated based on fourth sound data recorded during a urination process and a value related to a urination presence/absence corresponding to the fourth sound data, and the obtaining the urine volume determination value may include obtaining one or more adjusted first feature data by reflecting the urination presence/absence determination value to the one or more first feature data; and obtaining the urine volume determination value by using the one or more adjusted first feature data and the urine volume determination model.

The method of obtaining urination information may include obtaining a urination presence/absence classification value by using the urination presence/absence determination value, wherein the urination presence/absence classification value is either a urination section indication value or a non-urination section indication value, determined according to the urination presence/absence determination value; and obtaining an adjusted urine flow rate determination value by reflecting the urination presence/absence classification value to the urine flow rate determination value; and the estimated urine volume may be calculated by integrating the adjusted urine flow rate determination value over time.

The one or more first feature data may be generated by transforming the first sound data into a spectrogram and segmenting the spectrogram into a plurality of segmented spectrograms having a preset time length.

The obtaining a urine volume determination value may include obtaining a plurality of segmented urine volume determination value for each of the plurality of segmented spectrograms by inputting each of the plurality of segmented spectrograms into the urine volume determination model; and obtaining the urine volume determination value by adding the plurality of segmented urine volume determination value.

The method of obtaining urination information may further include when a time length of a last segmented spectrogram among the plurality of segmented spectrogram is shorter than the preset time length, padding on the last segmented spectrogram.

According to one embodiment of the present disclosure, a method of obtaining urination information may be provided, wherein the method of obtaining urination information includes obtaining one or more first feature data and one or more first relative feature data by using first sound data, wherein the first sound data reflect a sound of a urination process, and the first relative feature data comprise normalized value; obtaining a urine volume determination value by using the one or more first feature data and a pre-trained urine volume determination model, wherein the urine volume determination model is trained with a urine volume training data set, wherein the urine volume training data set comprises one or more second feature data generated based on second sound data recorded during a urination process and a value related to a urine volume corresponding to the second sound data; obtaining a relative urine flow rate determination value by using the one or more first relative feature data and pre-trained relative urine flow rate determination model, wherein the relative urine flow rate determination model is trained with a relative urine flow rate training data set, wherein the relative urine flow rate training data set comprises one or more second relative feature data generated based on third sound data recorded during a urination process and a value related to a relative urine flow rate corresponding to the third sound data; and obtaining a urine flow rate information by reflecting a ratio of an integral value calculated based on the relative urine flow rate determination value and the urine volume determination value to the relative urine flow rate determination value.

The integral value may be calculated by integrating the relative urine flow rate determination value over time.

The method of obtaining urination information may include obtaining a urination presence/absence determination value by using the one or more first feature data and pre-trained urination presence/absence determination model, wherein the urination presence/absence determination model is trained with a urination presence/absence training data set, wherein the urination presence/absence training data set comprises one or more third feature data generated based on fourth sound data recorded during a urination process and a value related to a urination presence/absence corresponding to the fourth sound data; obtaining a urination presence/absence classification value by using the urination presence/absence determination value, wherein the urination presence/absence classification value is either a urination section indication value or a non-urination section indication value, determined according to the urination presence/absence determination value; and obtaining an adjusted urine flow rate determination value by reflecting the urination presence/absence classification value to the urine flow rate determination value, the obtaining the urine volume determination value may include obtaining one or more adjusted first feature data by reflecting the urination presence/absence determination value to the one or more first feature data; and obtaining the urine volume determination value by using the one or more adjusted first feature data and the urine volume determination model, and the integral value may be calculated by integrating the adjusted urine flow rate determination value over time.

The one or more first feature data may be generated by transforming the first sound data into a spectrogram and segmenting the spectrogram into a plurality of segmented spectrograms having a preset time length, and the obtaining the urine volume determination value may include obtaining plurality of segmented urine volume determination value for each of the plurality of segmented spectrograms by inputting each of the plurality of segmented spectrograms into the urine volume determination model; and obtaining the urine volume determination value by adding the plurality of segmented urine volume determination value.

According to one embodiment of the present disclosure, a sound analysis system may be provided, wherein the sound analysis system includes a memory storing first sound data, pre-trained urine volume determination model and pre-trained urine flow rate determination model, wherein the first sound data reflect a sound of a urination process, the urine volume determination model is trained with a urine volume training data set comprising one or more first feature data generated based on second sound data recorded during a urination process and a value related to a urine volume corresponding to the second sound data, and the urine flow rate determination model is trained with a urine flow rate training data set comprising one or more second feature data generated based on third sound data recorded during a urination process and a value related to urine flow rate corresponding to the third sound data; and at least one processor, wherein the processor obtain one or more third feature data by using the first sound data, obtain a urine volume determination value by using the one or more third feature data and the urine volume determination model, obtain a urine flow rate determination value by using the one or more third feature data and the urine flow rate determination model, and obtain a urine flow rate information by reflecting a ratio of an estimated urine volume calculated based on the urine flow rate determination value and the urine volume determination value to the urine flow rate determination value.

According to one embodiment of the present disclosure, a sound analysis system may be provided, wherein the sound analysis system includes a memory storing first sound data, pre-trained urine volume determination model and pre-trained relative urine flow rate determination model, wherein the first sound data reflect a sound of a urination process, the urine volume determination model is trained with a urine volume training data set comprising one or more first feature data generated based on second sound data recorded during a urination process and a value related to a urine volume corresponding to the second sound data, and the relative urine flow rate determination model is trained with a relative urine flow rate training data set comprising one or more first relative feature data generated based on third sound data recorded during a urination process and a value related to relative urine flow rate corresponding to the third sound data; and at least one processor, wherein the processor obtain one or more second feature data and one or more second relative feature data by using the first sound data, obtain a urine volume determination value by using the one or more second feature data and the urine volume determination model, obtain a relative urine flow rate determination value by using the one or more second relative feature data and the relative urine flow rate determination model, and obtain a urine flow rate information by reflecting a ratio of an integral value calculated based on the relative urine flow rate determination value and the urine volume determination value to the relative urine flow rate determination value.

Hereinafter, a method of obtaining urination information and a device thereof according to an exemplary embodiment will be described.

1. Configuration of Urination Information Obtaining System 10

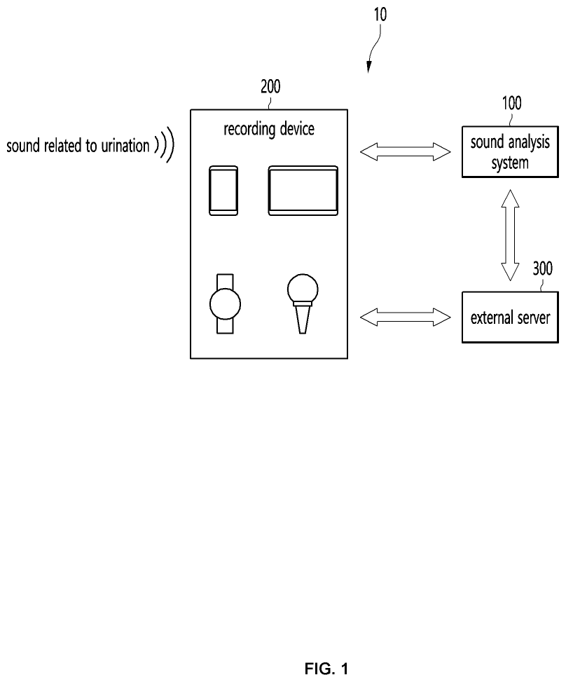

FIG. 1 is a view illustrating a urination information obtaining system 10 according to the exemplary embodiment.

The urination information obtaining system 10 according to the exemplary embodiment may obtain urination information by using data of a urination process. More specifically, referring to FIG. 1, the urination information obtaining system 10 may include a sound analysis system 100, a recording device 200, and an external server 300.

From the recording device 200, the sound analysis system 100 may obtain sound data of which the sound of the urination process is recorded and obtain urination information by using the obtained sound data. Alternatively, from the external server 300, the sound analysis system 100 may obtain sound data stored in the external server 300 and obtain urination information by using the obtained sound data.

Meanwhile, the sound analysis system 100 may obtain feature data from the sound data and obtain urination information by using the obtained feature data. The feature data may be data converted from the sound data by using feature values of the sound data, and the feature data may include data of the urination process.

For example, in sound data, the feature data may include at least one of a magnitude value of time band spectrum, a spectral centroid, a magnitude value of frequency band spectrum, a root mean square (RMS) value of frequency band, a magnitude value of spectrogram, a magnitude value of Mel-spectrogram, a Bispectrum Score (BGS), a Non-Gaussianity Score (NGS), Formants Frequencies (FF), a value of Log Energy (LogE), a Zero Crossing Rate (ZCR), a degree of Kurtosis (Kurt), and a Mel-frequency cepstral coefficient (MFCC). As an example, the feature data may be understood as data in a vector form, a matrix form, or other forms of feature values.

Meanwhile, the sound analysis system 100 may also receive feature data itself for sound data from the outside.

A specific process by which the sound analysis system 100 obtains urination information by using sound data and/or feature data will be described below.

The sound analysis system 100 may provide urination information obtained by analyzing sound data to the external server 300. That is, the sound analysis system 100 may obtain the urination information by analyzing the sound data of a urination process, the sound data being received from the outside, and output or provide the urination information to the outside.

The urination information may include a maximum flow rate during a urination process, an average flow rate, a urine volume, a start time point and end time point of urination, a urine flow time, a time to maximum urine flow rate, a urination time (with or without interruption time), and the like.

The recording device 200 may obtain sound data by recording sound related to urination.

The recording device 200 may obtain the sound data by recording the sound related to the urination. While worn on a person, the recording device 200 may record sound during a urination process, or may be placed in a space where urination occurs to record sound generated during the urination process.

Here, the sound data may be obtained by digitizing analog sound signals of the urination process. For example, the recording device 200 may include an analog to digital converter (ADC) module, and obtain sound data from the sound signals of the urination process by using specific sampling rates such as 8 kHz, 16 kHz, 22 kHz, 32 kHz, 44.1 kHz, 48 kHz, 96 kHz, 192 kHz, or 384 kHz.

The recording device 200 may obtain feature data from the obtained sound data. The feature data may be data converted from the sound data by using feature values of the sound data, and since the feature data has been described above, a redundant description will be omitted.

The recording device 200 may provide the obtained sound data to the sound analysis system 100 and/or the external server 300. To this end, the recording device 200 may perform wired and/or wireless data communication with the sound analysis system 100 and/or the external server 300.

Meanwhile, the recording device 200 may output urination information. For example, the recording device 200 may receive the urination information from the sound analysis system 100 and output the urination information to a user.

As an example, the recording device 200 may include a wearable device, equipped with a recording function, such as a smart watch, a smart band, a smart ring, and a smart neckless, or may include a smartphone, a tablet, a desktop, a laptop, a portable recorder, an installation-type recorder, or the like.

The external server 300 may store or provide various data. For example, the external server 300 may store sound data obtained from the recording device 200, and/or store urination information obtained from the sound analysis system 100. As another example, the external server 300 may obtain and store sound data obtained from the recording device 200, provide the sound data to the sound analysis system 100, store urination information obtained from the sound analysis system 100, and provide the urination information to the recording device 200.

The external server 300 may perform data processing by using the obtained urination information. As an example, the external server 300 may perform the data processing such as generating a urination-related chart or calculating statistical values by using the urination information, and may also process and convert the urination information into visual data such as changes in urination information by date.

Meanwhile, the above-described individual devices may be implemented as one device.

As an example, the sound analysis system 100 and the recording device 200 may be implemented as one device. In this case, the sound analysis system 100 by itself may include a module having a recording function and obtain sound data.

Alternatively, components of the sound analysis system 100 may be built into the recording device 200 so that the recording device 200 by itself may provide a function for analyzing sound data.

As another example, the sound analysis system 100 may be implemented as one device with the external server 300.

In addition, the operations of the above-described individual devices may be performed by other entities. As an example, the recording device 200 may obtain sound data and convert the obtained sound data into feature data. The recording device 200 may transmit the feature data to the sound analysis system 100, and the sound analysis system 100 may obtain urination information by using the received feature data.

2. Configuration of Sound Analysis System 100

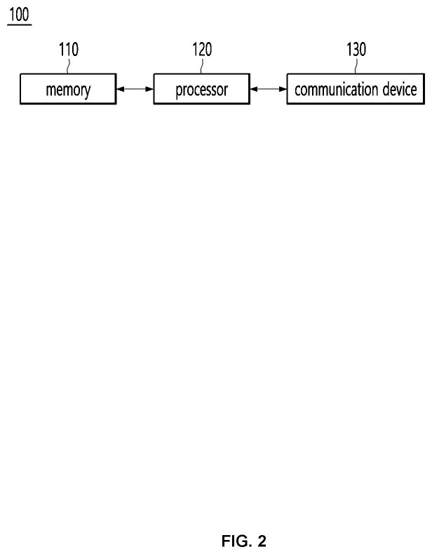

FIG. 2 is a block diagram illustrating a configuration of the sound analysis system 100 according to the exemplary embodiment.

Referring to FIG. 2, the sound analysis system 100 may include a memory 110, a processor 120, and a communication device 130.

The memory 110 may store various processing programs, parameters for the processing programs, result data of such processing, and the like. For example, the memory 110 may store instructions for the operation of the processor 120 to be described below, and store a urine volume determination module, a urination presence/absence determination module, a urine flow rate determination module, and/or a relative urine flow rate determination module, which are used to obtain urination information. In addition, the memory 110 may also store the urine volume determination model, urination presence/absence determination model, urine flow rate determination model, and/or relative urine flow rate determination model, which are used to obtain the urination information.

The determination models and/or determination modules may be models and/or modules, which are trained by the processor 120. Without being limited thereto, the determination models and/or the determination modules may also be models and/or modules, which have been trained in advance and received from the outside. Specific details about the determination models and determination modules will be described below.

The memory 110 may also store a training data set for training the determination models used to obtain urination information. The training data may include sound data obtained during a urination process, feature data generated on the basis of the sound data, and/or urine volumes, urine flow rate values, urination presence/absence values, and the like of the urination process corresponding to the sound data.

The memory 110 may store sound data and/or feature data, which are analysis targets to obtain urination information, such as urine volumes, urine flow rate values, and/or urination presence/absence values. Since the feature data has been described above, a redundant description will be omitted.

The memory 110 may be implemented with a non-volatile semiconductor memory, a hard disk, a flash memory, a random-access memory (RAM), a read-only memory (ROM), an electrically erasable programmable read-only memory (EEPROM), other tangible non-volatile recording media, or the like

The processor 120 may operate according to instructions stored in the memory 110. According to the exemplary embodiment, the processor 120 may perform preprocessing on sound data stored in the memory 110, and the processor 120 may obtain feature data by using the sound data.

The processor 120 may obtain urination information from sound data or feature data by using the urine volume determination module, the urination presence/absence determination module, the urine flow rate determination module, and/or the relative urine flow rate determination module, which are stored in the memory 110. Specifically, the process of obtaining the urination information will be described below.

The processor 120 may train the urine volume determination model, the urination presence/absence determination model, the urine flow rate determination model, and/or the relative urine flow rate determination model by using a training data set stored in the memory 110. The specific model training process will be described below.

The processor 120 may obtain urination information by performing a method of obtaining urination information to be described below, and may also perform data processing on the obtained urination information.

Unless otherwise specified, various operations or steps of obtaining urination information, which are disclosed as the exemplary embodiments below, may be interpreted as being performed either by the processor 120 of the sound analysis system 100 or under the control of the processor 120.

Meanwhile, the processor 120 may be implemented with a central processing unit (CPU), a graphics processing unit (GPU), a digital signal processor (DSP), a state machine, an application-specific integrated circuit (ASIC), a radio-frequency integrated circuit (RFIC), a combination thereof, and the like.

The communication device 130 may transmit or receive data and/or information to or from the outside through wired and/or wireless communication. The communication module 130 may perform bidirectional or unidirectional communication.

Through the communication device 130, the sound analysis system 100 may receive sound data and/or feature data from the outside, and may also receive measured data of a urination process (e.g., measured urine volumes, measured urine flow rate values, etc.). Meanwhile, through the communication device 130, the sound analysis system 100 may also receive a pre-trained urine volume determination model, a pre-trained urination presence/absence determination model, a pre-trained urine flow rate determination model, and/or a pre-trained relative urine flow rate determination model.

The sound analysis system 100 may transmit the obtained urination information to the recording device 200 and/or the external server 300 through the communication device 130. Without being limited thereto, the sound analysis system 100 may also communicate with other external devices through the communication device 130.

3. Method of Obtaining Urination Information by Using Urine Volume Determination Model

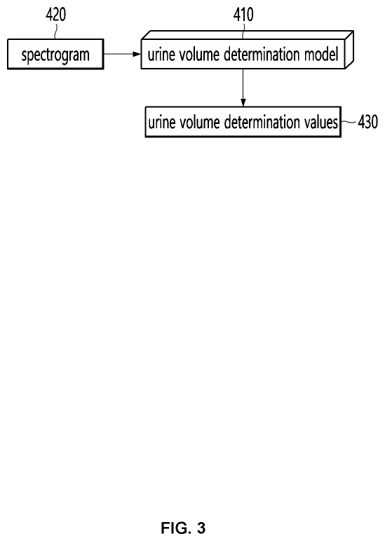

FIG. 3 is a view illustrating a urine volume determination model 410 according to the exemplary embodiment.

The urine volume determination model 410 may be a model trained to receive data of a urination process and output urine volume data. The urine volume data may include values regarding urine volumes during the urination process.

Referring to FIG. 3, data of a urination process may be a spectrogram 420, and urine volume data may be urine volume determination values 430. The urine volume determination model 410 may output the urine volume determination values 430 predicted during a urination process corresponding to the input spectrogram 420.

The urine volume determination model 410 may refer to a model trained by using machine learning. Here, the machine learning may be understood as a comprehensive concept including an artificial neural network and further including deep learning. Such an algorithm may use at least any one of k-nearest neighbors, linear regression, logistic regression, a support vector machine (SVM), a decision tree, a random forest, or a neural network. Here, as the neural network, at least one from among an artificial neural network (ANN), a time delay neural network (TDNN), a deep neural network (DNN), a convolution neural network (CNN), a recurrent neural network (RNN), or a long short-term memory (LSTM) may be selected.

Sound data may generally include amplitude values over time. Through processing, the sound data may be converted into spectrum data including magnitude values depending on frequencies.

Here, the spectrum data may be obtained by using Fourier transform (FT), Fast Fourier transform (FFT), Discrete Fourier transform (DFT), and Short-time Fourier transform (STFT).

A spectrogram 420 may be obtained by using the above-described sound data and spectrum data. Here, the spectrogram 420 may be a Mel-spectrogram image to which a Mel-scale is applied. The values of the Mel-spectrogram image may be understood as a set of data in a matrix form considering a time axis and a frequency axis.

Meanwhile, a component described as a spectrogram in the present disclosure is intended to facilitate understanding of the content, and even though not described separately in the present disclosure, the component described as the spectrogram may naturally be included in the technical idea of the present disclosure even in a case of realizing the component as sound data itself or other feature data obtained from the sound data.

That is, the urine volume determination model 410 may also output urine volume determination values by using sound data itself or other feature data obtained from the sound data. Since the content related to the feature data has been described above, a redundant description will be omitted.

The urine volume determination model 410 may be trained by using training data, and the training data may include sound data of a urination process and a urine volume of the urination process.

The sound data of the urination process may be data obtained by recording sound generated during the urination process.

A urine volume of a urination process may be a measured urine volume actually measured during the urination process corresponding to sound data.

In addition, for the purpose of enhancing training data, a urine volume of a urination process (i.e., training data) may include a virtual urine volume derived from the measured urine volume. Hereinafter, a component described as a measured urine volume may be understood to include a derived urine volume from the measured urine volume.

As an example, a measured urine volume may be calculated by using a urine volume collected during a urination process.

As another example, a measured urine volume may be calculated by using a toilet weight change before and after a urination process. In this case, a separate device for measuring a toilet weight may be provided.

As a yet another example, a measured urine volume may be calculated by using a weight change of a person's body before and after a urination process. Specifically, a urine volume may be determined by multiplying a difference between the person's weight before the urination process and the person's weight after the urination process by a predetermined coefficient. In this case, the measured urine volume may be obtained by measuring the weight change of the person by using a general scale, so there is a strong point in that the measured urine volume may be obtained more easily.

Without being limited thereto, various methods may be used to obtain a urine volume during a urination process.

Meanwhile, the urine volume determination model may be trained by using training data on which separate preprocessing has been performed.

As preprocessing, filtering for removing noise may be performed on sound data. Here, the filtering may refer to a process of excluding noise-related data from the sound data.

As preprocessing, cropping of a section corresponding to a urination section may be performed in sound data. The section corresponding to the urination section may be determined by a person directly listening to the sound data, and may also be determined by using separately a trained determination model.

As preprocessing, a task of converting sound data into separate feature data may be performed. The feature data may be converted from the sound data by using feature values of the sound data, and the feature data may include data of a urination process.

In sound data, the feature data may include at least one of a magnitude value of time band spectrum, a spectral centroid, a magnitude value of frequency band spectrum, an RMS value of frequency band, a magnitude value of spectrogram, a magnitude value of Mel-spectrogram, BGS, NGS, FF, LogE, ZCR, Kurt, and MFCC.

Various conversion methods may be performed on sound data depending on the type of feature data to be converted. For example, in a case where feature data to be converted is a spectrum, sound data may be converted into spectrum data having a frequency axis. As another example, in a case where feature data to be converted is a spectrogram, sound data may be converted into a spectrogram having a time axis and a frequency axis.

In a case when there are multiple types of feature data to be converted, two or more pieces of feature data may be generated by using identical sound data, and in this case, the two or more pieces of the feature data may also not be identical to each other as well.

Hereinafter, for convenience of description, a case where feature data is a spectrogram is mainly described. However, the technical idea of the present disclosure is not limited thereto, and may be similarly applied even when the feature data is in other forms.

As preprocessing, a spectrogram may be segmented by a predetermined time length. As an example, the predetermined time length may be a value between five seconds and ten seconds.

A spectrogram may be segmented so that segmented sections do not overlap each other, and lengths of respective segmented spectrograms may be the same as each other.

Meanwhile, the segmented spectrograms may also be generated so that sections thereof overlap each other. Specifically, a first segmented spectrogram set may be obtained by first segmenting a spectrogram by a predetermined time length so that segmented sections do not overlap each other. In addition, as many values of zeros as a time length shorter than a time length predetermined in a start section of the spectrogram are added to obtain a second spectrogram, and the second spectrogram is segmented by the predetermined time length so that the segmented sections do not overlap each other, whereby a second segmented spectrogram set may be obtained. In this case, for respective segmented spectrograms, when the first segmented spectrogram set and the second segmented spectrogram set are viewed together, other segmented spectrograms having sections overlapping each other may exist. Meanwhile, a urine volume corresponding to the sum of the first segmented spectrogram set and the second segmented spectrogram set may be a value twice a measured urine volume.

Without being limited thereto, a second spectrogram may also be obtained by cropping as many sections as a time length shorter than a time length predetermined in a start section of a spectrogram. In this case, in the sections to be cropped, sections may be cropped so that there is no value corresponding to sound generated during a urination process left.

The above-described segmented spectrogram sets different from each other have been described as that two sets are generated, but it is not limited thereto, and a plurality of segmented spectrogram sets may be generated by varying a time length for adding or cutting.

In a case when the total time length for a spectrogram is not an integer multiple of a predetermined time length, a length of a last segmented spectrogram may be shorter than the predetermined time length. In this case, as preprocessing, padding may be additionally performed on the last segmented spectrogram.

Padding may refer to a task of adding determined data to a section having no data. As an example, in a case where the last segmented spectrogram does not satisfy the predetermined time length, zero padding may be performed to set values for a short time section to values of zeros.

Meanwhile, the preprocessing for the above-described training data is not essential, and some processes of the above-described preprocessing processes may be omitted, or the preprocessing itself may also not be performed at all. In a case when the task of converting sound data into separate feature data as preprocessing is omitted, the segmenting by the predetermined time length and the padding may also be performed on sound data itself as preprocessing.

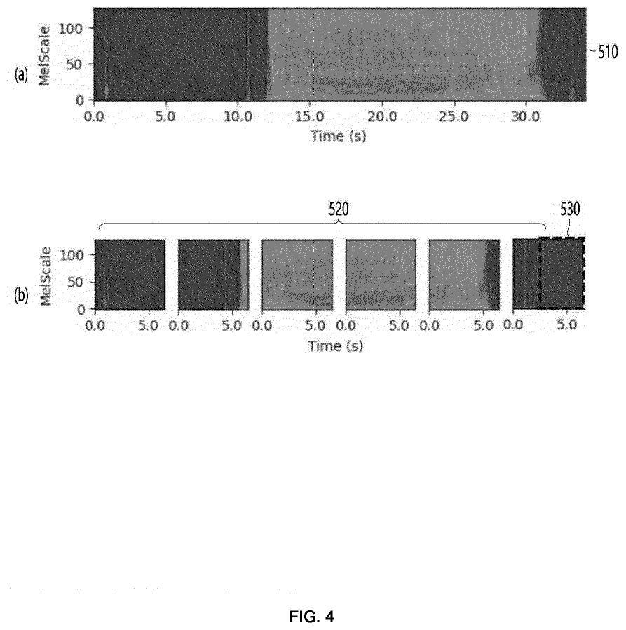

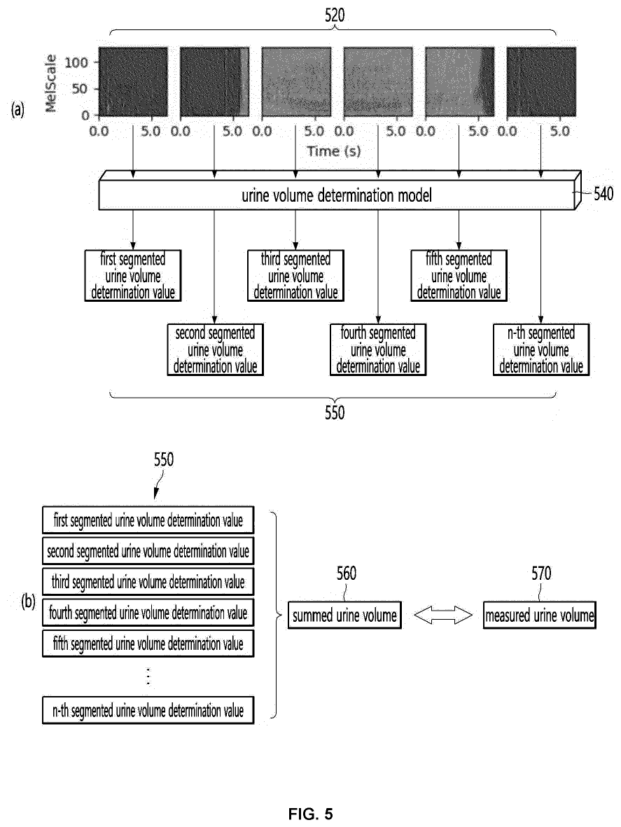

Specifically, a training process of the urine volume determination model will be described with reference to FIGS. 4 and 5.

FIGS. 4 and 5 are views illustrating a training process of a urine volume determination model 540 according to the exemplary embodiment.

FIG. 4(a) shows a spectrogram 510 converted from sound data recorded during a urination process.

FIG. 4(b) shows a plurality of segmented spectrograms 520 in which the spectrogram 510 is segmented by 6.4 seconds, which is a predetermined time length.

FIG. 4 shows that the spectrogram 510 is segmented into six segmented spectrograms. However, this is according to an example, and the number of spectrogram segments and the predetermined time length thereof are not limited thereto.

Referring to FIG. 4(b), a time length of the first spectrogram 510 is not an integer multiple of the predetermined time length of 6.4 seconds, so a time length of a last segmented spectrogram may be shorter than the predetermined time length. In this case, as described above, padding may be performed on a short time section 530. As an example, zero padding for adding values of zeros may be performed.

FIG. 5(a) shows that a urine volume determination model 540 uses segmented spectrograms 520 to output segmented urine volume determination values 550 corresponding to the segmented spectrogram 520. As an example, the urine volume determination model 540 may output a first segmented urine volume determination value corresponding to a first segmented spectrogram, may output a second segmented urine volume determination value corresponding to a second segmented spectrogram, . . . , and may output an n-th segmented urine volume determination value corresponding to an n-th segmented spectrogram.

As shown in FIG. 5(b), the urine volume determination model 540 may be trained so that a urine volume 560, which is the sum of a plurality of segmented urine volume determination values 550, corresponds to a measured urine volume 570 labeled in the spectrogram 510. Although not shown in FIG. 5, in a case where two sets of segmented spectrograms are generated by using the above-described method so that sections thereof overlap each other, the urine volume determination model may be trained so that the urine volume, which is the sum of the plurality of segmented urine volume determination values, corresponds to a value twice a measured urine volume labeled in a spectrogram. Meanwhile, in a case where m sets of segmented spectrograms are generated by using the above-described method so that sections thereof overlap each other, the urine volume determination model may be trained so that a urine volume, which is the sum of the plurality of segmented urine volume determination values, corresponds to a value m times a measured urine volume labeled in a spectrogram.

The urine volume determination model may be trained through various methods such as supervised training, unsupervised training, reinforcement training, and imitation training. As an example, the urine volume determination model 540 may be trained by comparing the summed urine volume 560 and the measured urine volume 570 and back-propagating an error thereof.

Meanwhile, the urine volume determination model 540 has been described as outputting the urine volume determination values 550, but it is not limited thereto, and the urine volume determination model may also be configured to output other feature values related to a urine volume.

Meanwhile, the urine volume determination model 540 has been described as being trained by using the segmented spectrograms 520 obtained by segmenting the spectrogram 510 and the summed urine volume 560 obtained by adding the segmented urine volume determination values 550, but it is not limited thereto. The urine volume determination model may be configured to output one urine volume determination value by using the unsegmented spectrogram itself, and may also be configured to be trained by using the one urine volume determination value and measured urine volume, which are output.

Meanwhile, the urine volume determination model has been described as outputting urine volume determination values by using a spectrogram, but it is not limited thereto, and the urine volume determination model may also be configured to output urine volume determination values by using other feature data or sound data itself other than the spectrogram. Since the feature data has been described above, a redundant description will be omitted.

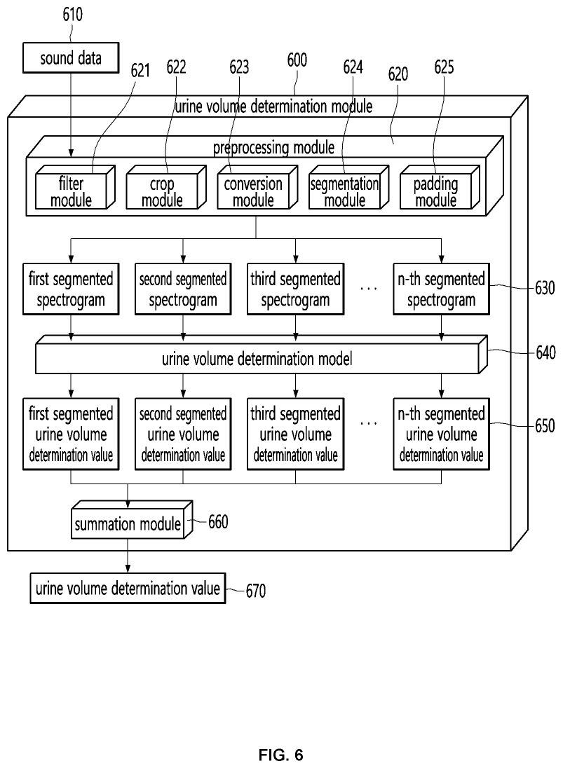

FIG. 6 is a view illustrating a configuration of a urine volume determination module 600 according to the exemplary embodiment.

Referring to FIG. 6, the urine volume determination module 600 may output a urine volume determination value 670 by using sound data 610. Without being limited thereto, the urine volume determination module 600 may also be configured to output a urine volume determination value by using a spectrogram or other feature data. The urine volume determination value may refer to urine volume data, and the urine volume data may include a value regarding a urine volume during a urination process.

The urine volume determination module 600 according to an example may include a preprocessing module 620, a urine volume determination model 640, and a summation module 660.

The preprocessing module 620 may include a filter module 621, a crop module 622, a conversion module 623, a segmentation module 624, and/or a padding module 625.

The filter module 621 may remove noise-related data from the sound data 610. The filter module 621 may include a high-pass filter, a low-pass filter, a band-pass filter, etc.

The crop module 622 may crop a section corresponding to a urination section in the sound data. As an example, when a user inputs information on urination start and end time points of sound data together with the sound data into the urine volume determination module 600, the cropping module 622 may crop the sound data between the urination start and end time points. Meanwhile, the urination start and end time points of sound data may also be determined by a person listening to the sound data, but it is not limited thereto, and the urination start and end time points may also be determined by using a trained urination time point determination model.

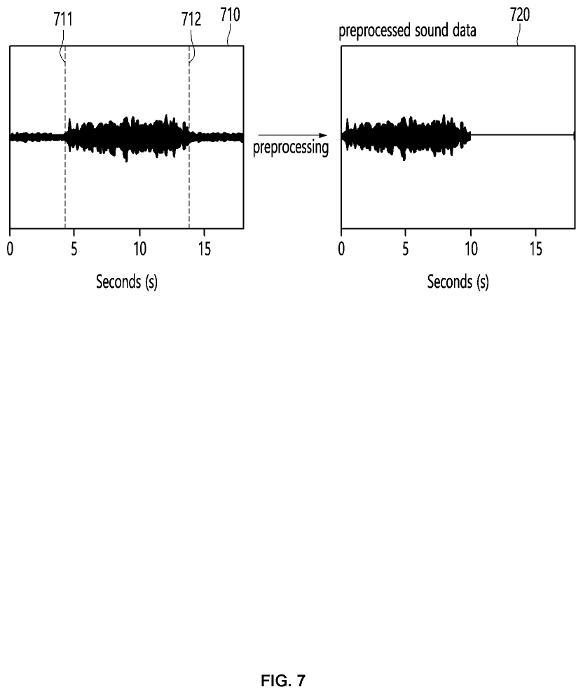

The crop module 622 may also process sound data by moving the cropped sound data in a time dimension so that a urination start time point starts from zero seconds of the sound data. Specific details will be described with reference to FIG. 7.

FIG. 7 is a view illustrating sound data preprocessing according to the exemplary embodiment.

Referring to FIG. 7, when information on a urination start time 711 and a urination end time 712 together with sound data 710 is input, the urine volume determination module 600 may obtain, from the sound data 710, preprocessed sound data 720 on which cropping between the urination start and end time points is performed through the crop module 622.

Referring back to FIG. 6, in a case where the urine volume determination model 640, which will be described below, is a model trained to output urine volume determination values by using a spectrogram rather than sound data, the conversion module 623 included in the preprocessing module 620 may convert the sound data into a spectrogram.

Without being limited thereto, the conversion module 623 may convert the sound data into other feature data in a case where the urine volume determination model 640 is trained to output urine volume determination values by using other feature data. Since the types of feature data have been described above, a redundant description will be omitted.

The segmentation module 624 may segment the spectrogram, which is converted by the conversion module 623, by a predetermined time length. In this case, the segmentation module 624 may segment the spectrogram so that respective segmented spectrograms do not overlap each other. Without being limited thereto, the segmentation module may also generate a segmented spectrogram set whose sections overlap each other as described above. A predetermined time length may be the same as a predetermined time length considered when the training data of the urine volume determination model 640 is processed. As an example, the predetermined time length may be a value between five seconds and ten seconds.

Meanwhile, in a case where the urine volume determination model 640 is a model trained to output urine volume determination values by using sound data itself, the segmentation module 624 may be configured to segment the sound data 610 by a predetermined time length.

The padding module 625 may perform padding of predetermined data in a last segmented spectrogram in a case where a length of the last segmented spectrogram among the segmented spectrograms segmented by the segmentation module 624 is shorter than a predetermined time length due to the total length of the spectrogram that is not an integer multiple of the predetermined time length. As an example, the padding module 625 may perform zero padding for adding as many values of zeros as a time length required in order for the last segmented spectrogram to satisfy the predetermined time length.

Meanwhile, in the case where the urine volume determination model 640 is a model trained to output urine volume determination values by using the sound data itself, the padding module 625 may also perform padding of predetermined data in the last segmented sound data when a length of the last segmented sound data among the segmented sound data segmented by a predetermined time length is shorter than the predetermined time length. As an example, the padding module 625 may perform zero padding for adding as many values of zeros as a time length required in order for the last segmented sound data to satisfy a predetermined time length.

The urine volume determination model 640 is a model trained to receive an input of data of a urination process and output data including values about a urine volume during the urination process. As an example, the urine volume determination model 640 may be a model trained to output urine volume determination values by using a spectrogram, and may be a model trained with the above-described method of training the urine volume determination model. Without being limited thereto, the urine volume determination model 640 may also be a model trained to output data including values related to urine volumes by using sound data itself or other feature data.

Referring to FIG. 6, the urine volume determination model 640 may output first to n-th segmented urine volume determination values 650, which correspond to respective segmented spectrograms, by using first to n-th segmented spectrograms 630 segmented by the segmentation module 624.

The summation module 660 may add the first to n-th segmented urine volume determination values 650 and output a urine volume determination value 670 corresponding to the sound data 610. Meanwhile, in a case where the segmentation module 624 is configured to generate, from a spectrogram, a segmented spectrogram set whose sections overlap each other, the summation module 660 may also output a urine volume determination value obtained by dividing the sum of the first to n-th segmented urine volume determination values by the number of segmented spectrogram sets.

In a case when the urine volume determination model 640 is a model trained to output other feature values related to a urine volume, the summation module 660 may also output a urine volume determination value by performing processing to sum segmented feature values output by the urine volume determination model 640 and convert a summed value into a urine volume. As an example, the summation module 660 may be configured to output a urine volume determination value by reflecting a specific value in a value obtained by adding the segmented feature values.

As described above, the urine volume determination module 600 segments a spectrogram by a predetermined time length, obtains segmented urine volume determination values by using segmented spectrograms, and outputs a total urine volume determination value by adding the segmented urine volume determination values. Even when sound data of various lengths is input, the urine volume determination model may determine a urine volume by using the spectrogram having a predetermined length thereof, so there is an effect of reducing the influence of the total length of the sound data on the accuracy of a urine volume determination result.

Meanwhile, the urine volume determination module 600 has been described as receiving an input of the sound data 610 and outputting the urine volume determination value 670, but is not limited thereto. The urine volume determination module may also be configured to receive an input of a spectrogram or other feature data itself and output a urine volume determination value. In this case, the above-described crop module 622 may be configured to crop between urination start and end time points in the spectrogram, and the conversion module 623 may not be included in the preprocessing module 620.

Meanwhile, the urine volume determination module 600 is not required to include all the above-described filter module 621, crop module 622, conversion module 623, segmentation module 624, and padding module 625. As an example, the urine volume determination module 600 may include merely some modules from among the filter module 621, crop module 622, conversion module 623, segmentation module 624, and padding module 625, or may also not include the preprocessing module 620 itself. In a case when the urine volume determination module 600 does not include the segmentation module 624, the urine volume determination module 600 may also not include the summation module 660. In the case where the urine volume determination module 600 does not include the preprocessing module 620, the urine volume determination module 600 may be configured to allow the urine volume determination model 640 to output urine volume determination values by using the sound data 610 itself.

4. Method of Obtaining Urination Information by Using Urine Volume Determination Module and Urination Presence/Absence Determination Module

In order to obtain highly accurate urine volume information, the urination presence/absence determination model may be used together with the urine volume determination model.

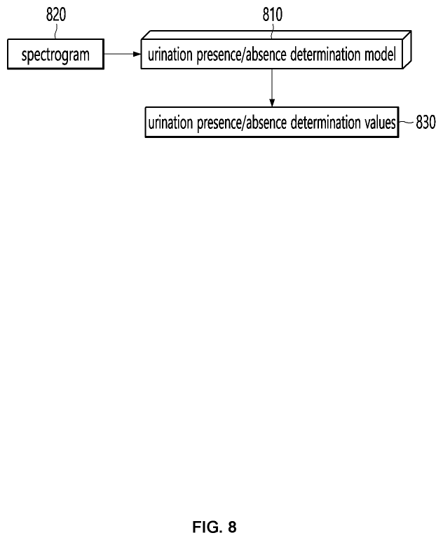

FIG. 8 is a view illustrating a urination presence/absence determination model 810 according to the exemplary embodiment.

The urination presence/absence determination model 810 may be a model trained to receive an input of data of a urination process and output urination presence/absence data. The urination presence/absence data may include values for classifying a urination section or a non-urination section during a urination process. The urination presence/absence data may be understood as vector data, matrix data, or data in other formats.

Referring to FIG. 8, data of a urination process may be a spectrogram 820, and urination presence/absence data may be urination presence/absence determination values 830. The urination presence/absence determination model 810 may output urination presence/absence determination values 830 over time predicted for the urination process corresponding to the input spectrogram 820.

A urination presence/absence determination value 830 may be a urination presence/absence probability value based on whether a urination process corresponding to the spectrogram 820 is in a urination section or a non-urination section over time or a classification value for classifying urination and non-urination. The probability value may be a value between 0 and 1, and the classification value may be a value indicating the urination section (e.g., a value of 1) or a value indicating the non-urination section (e.g., a value of 0).

In a urination section, a probability value may have a value close to 1, and in a non-urination section, a probability value may have a value close to 0. Meanwhile, in the urination section, a classification value may have a value indicating the urination section, and in the non-urination section, a classification value may have a value indicating the non-urination section.

Meanwhile, a classification value may also be a value determined on the basis of a urination presence/absence determination value. As an example, the classification value may be determined as a value indicating a urination section in a case where a urination presence/absence determination value is greater than or equal to a reference value, and may be determined as a value indicating a non-urination section in a case where a urination presence/absence determination value is less than the reference value. It is not limited thereto, and the opposite case is also applicable.

The urination presence/absence determination model 810 may refer to a model trained by using machine learning. Here, the machine learning may be understood as a comprehensive concept that includes an artificial neural network and further includes deep learning. As an algorithm thereof, at least any one of k-nearest neighbors, linear regression, logistic regression, a support vector machine, a decision tree, a random forest, or a neural network may be used. Here, at least one of ANN, TDNN, DNN, CNN, RNN, or LSTM may be selected as the neural network.

A urination classification value that includes a value indicating a urination section (e.g., a value of 1) or a value indicating a non-urination section (e.g., a value of 0) may be generated by applying a step function, a sigmoid function, a Softmax function, or the like to a urination presence/absence determination value.

Since the content related to the spectrogram used by the urination presence/absence determination model 810 has been described above in the part describing the urine volume determination model, a redundant description will be omitted.

Meanwhile, the urination presence/absence determination model 810 may also output urination presence/absence determination values by using sound data itself or other feature data obtained from the sound data. Since the content related to the feature data has been described above, a redundant description will be omitted.

The urination presence/absence determination model may be trained by using training data, and the training data may include sound data of a urination process and urination presence/absence values of the urination process.

Since the content related to the sound data of the urination process has been described above in the part describing the urine volume determination model, a redundant description will be omitted.

Urination presence/absence values of a urination process may be measured urination presence/absence values determined during a urination process corresponding to sound data.

In addition, for the purpose of enhancing training data, the urination presence/absence values of the urination process (i.e., the training data) may include virtual urination presence/absence values derived from the measured urination presence/absence values. Hereinafter, a component described as a measured urination presence/absence value may be understood as including a urination presence/absence value derived from the measured urination presence/absence value.

As an example, in a measured urination presence/absence value, a person may directly listen to sound data, and assign a urination value to a urination section and a non-urination value to a non-urination section for the sound data. Here, the urination value may be 1 and the non-urination value may be 0.

As another example, a measured urination presence/absence value may be obtained on the basis of data on a toilet weight change obtained during a urination process. Specifically, after setting two pieces of data so that a time domain of sound data and a time domain of data on a toilet weight change, which are obtained during a urination process, are the same as each other, a urination value may be assigned to a section of sound data corresponding to a section where a toilet weight change is detected, and a non-urination value may be assigned to a section of sound data corresponding to a section where a toilet weight change is not detected, Here, the urination value may be 1 and the non-urination value may be 0.

Without being limited thereto, various methods may be used to obtain urination presence/absence values during a urination process.

Meanwhile, the urination presence/absence determination model may also be trained by using training data on which separate preprocessing has been performed.

As preprocessing, filtering for removing noise may be performed on sound data. Since the filtering has been described above in the part describing the urine volume determination model, a redundant description will be omitted.

As preprocessing, cropping of a section corresponding to a urination section in sound data may be performed. Since the cropping of the section corresponding to the urination section has been described in the part describing the urine volume determination model, a redundant description will be omitted.

As preprocessing, a task of converting sound data into separate feature data may be performed. As an example, the feature data may be a spectrogram, and since converting into the feature data has been described above in the part describing the urine volume determination model, a redundant description will be omitted.

As preprocessing, a spectrogram may be segmented by a predetermined time length. The predetermined time length may be a value between two seconds and ten seconds. The spectrogram may be segmented so that segmented sections thereof do not overlap each other, and lengths of respective segmented spectrograms may be the same as each other. Without being limited thereto, and the spectrogram may also be segmented so that some sections of the respective segmented spectrograms overlap adjacent other segmented spectrograms.

As preprocessing, measured urination presence/absence values may also be segmented by a predetermined time length so that a spectrogram corresponds to the segmented time sections. The segmented measured urination presence/absence values, which are corresponding to the segmented spectrograms, may have consecutive values (e.g., 0, 0, 1, 1, 1, 0, etc.) over time while in the segmented sections. Values in a specific time section and included in the segmented measured urination presence/absence values may correspond to values in the same time section in the corresponding segmented spectrograms.

In a case when the total time length for a spectrogram and measured urination presence/absence values is not an integer multiple of a predetermined time length, respective lengths of a last segmented spectrogram and a last segmented measured urination presence/absence value may be shorter than the predetermined time length. In this case, as preprocessing, padding may be further performed on the last segmented spectrogram and the last segmented measured urination presence/absence value. Since the padding has been described above in the part describing the urine volume determination model, a redundant description will be omitted.

Meanwhile, preprocessing for the above-described training data is not essential, and some of the above-described preprocessing processes may be omitted or the preprocessing itself may also not be performed. In a case when the task of converting sound data into separate feature data as preprocessing is omitted, the segmenting by the predetermined time length and the padding may also be performed on sound data itself as preprocessing.

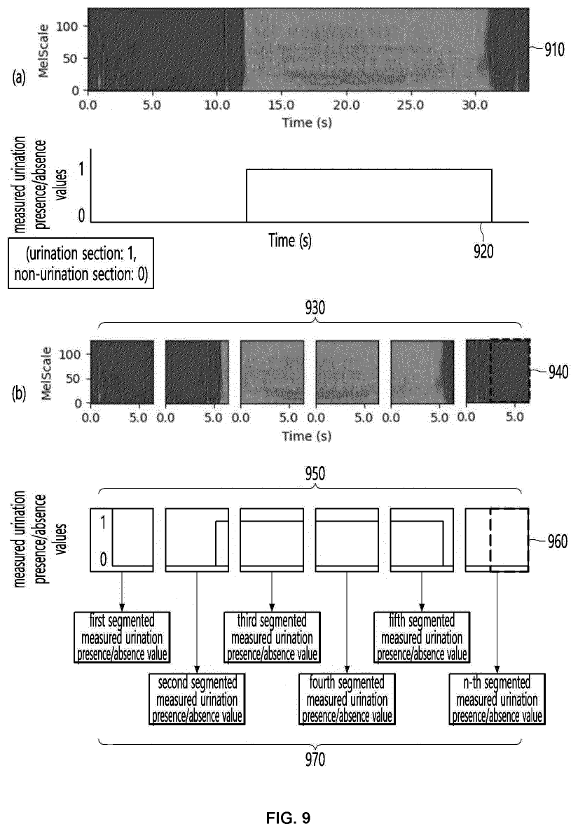

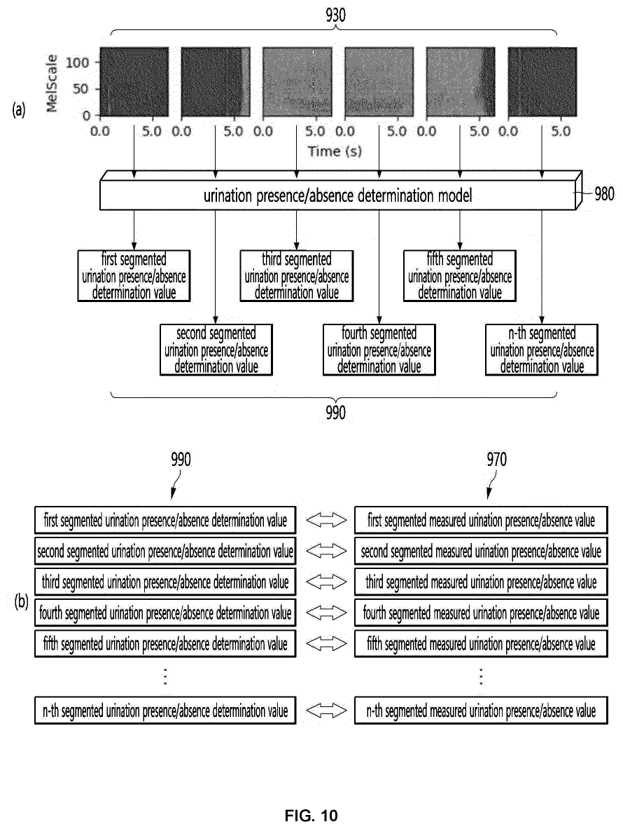

Specifically, a training process of the urination presence/absence determination model will be described with reference to FIGS. 9 and 10.

FIGS. 9 and 10 are views illustrating a training process of a urination presence/absence determination model 980 according to the exemplary embodiment.

FIG. 9(a) shows a spectrogram 910 converted from sound data recorded during a urination process and measured urination presence/absence values 920 corresponding to the sound data.

FIG. 9(b) shows segmented spectrograms 930 obtained by segmenting the spectrogram 910 by a predetermined time length of 6.4 seconds, and segmented measured urination presence/absence values 950 obtained by segmenting the measured urination presence/absence values 920 by the predetermined time length of 6.4 seconds.

In FIG. 9, the spectrogram 910 is segmented into six segmented spectrograms and the measured urination presence/absence values 920 are segmented into six segmented measured urination presence/absence values, but this is according to an example. The number of segmented spectrograms, the number of segmented measured urination presence/absence values, and the predetermined time length are not limited thereto.

Referring to FIG. 9(b), each of time lengths of the first spectrogram 910 and the measured urination presence/absence values 920 is not an integer multiple of the predetermined time length of 6.4 seconds, so a time length of a last segmented spectrogram and a time length of a last segmented measured urination presence/absence value may be shorter than the predetermined time length. In this case, as described above, padding may be performed on short time sections 940 and 960. As an example, zero padding for adding values of zeros may be performed. Consequently, it is possible to obtain segmented measured urination presence/absence values 970 having the same time lengths as each other and corresponding to respective segmented spectrograms 930 having the same time lengths as each other. The segmented measured urination presence/absence values 970 may have consecutive values over time while in segmented time sections.

FIG. 10(a) shows that the urination presence/absence determination model 980 outputs segmented urination presence/absence determination values 990 corresponding to the segmented spectrograms 930 by using the segmented spectrograms 930. Here, the segmented urination presence/absence determination values 990 may be urination presence/absence probability values or urination presence/absence classification values. As an example, the urination presence/absence determination model 980 may output a first segmented urination presence/absence determination value corresponding to a first segmented spectrogram, output a second segmented urination presence/absence determination value corresponding to a second segmented spectrogram, . . . , and output an n-th segmented urination presence/absence determination value corresponding to an n-th segmented spectrogram.

As in FIG. 10(b), the urination presence/absence determination model 980 may be trained so that the respective segmented urination presence/absence values 990 correspond to the segmented measured urination presence/absence values 970.

The urination presence/absence determination model 980 may be trained through various methods such as supervised training, unsupervised training, reinforcement training, and imitation training. As an example, the urination presence/absence determination model 980 may be trained by comparing the segmented urination presence/absence determination values 990 with the segmented measured urination presence/absence values 970 and backpropagating an error thereof.

Meanwhile, it is described that the urination presence/absence determination model 980 is trained by using the segmented spectrograms 930 obtained by segmenting the spectrogram 910 and the segmented measured urination presence/absence values 970 obtained by segmenting the measured urination presence/absence values 920, but it is not limited thereto. The urination presence/absence determination model may be configured to output a urination presence/absence determination value for an entire section by using an unsegmented spectrogram itself, and may also be configured to be trained by using a urination presence/absence determination value for the entire section and a measured urination presence/absence value, which are output.

Meanwhile, the urination presence/absence determination model has been described as outputting the urination presence/absence determination values by using a spectrogram, but it is not limited thereto, and the urination presence/absence determination model may be configured to output urination presence/absence determination values by using other feature data or sound data itself other than the spectrogram. Since the feature data has been described above, a redundant description will be omitted.

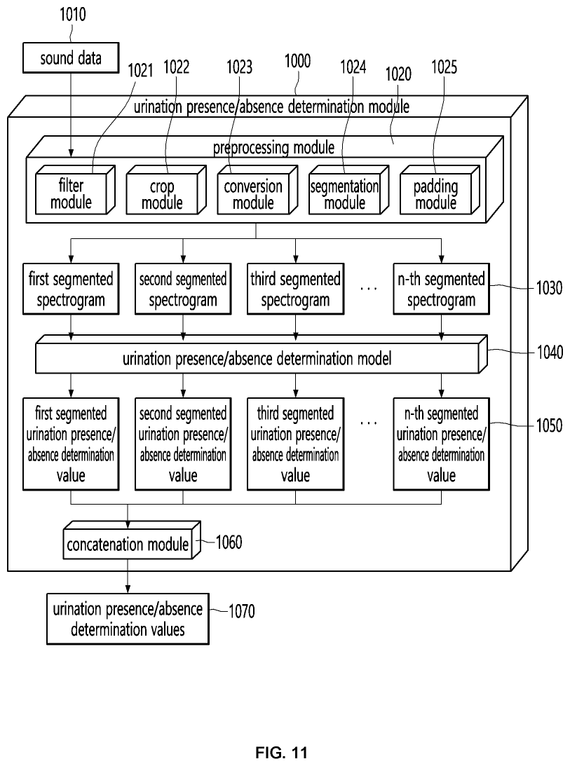

FIG. 11 is a view illustrating a configuration of a urination presence/absence determination module 1000 according to the exemplary embodiment.

Referring to FIG. 11, the urination presence/absence determination module 1000 may output urination presence/absence determination value 1070 by using sound data 1010. Without being limited thereto, the urination presence/absence determination module 1000 may be configured to output urination presence/absence determination values by using a spectrogram or other feature data. The urination presence/absence determination values may refer to urination presence/absence data, and the urination presence/absence data may include a value for classifying a urinary section or a non-urinary section during a urination process.

The urination presence/absence determination module 1000 according to one example may include a preprocessing module 1020, a urination presence/absence determination model 1040, and a concatenation module 1060.

The preprocessing module 1020 may include a filter module 1021, a crop module 1022, a conversion module 1023, a segmentation module 1024, and/or a padding module 1025.

The filter module 1021, crop module 1022, conversion module 1023, and padding module 1025 may operate similarly to the filter module 621, crop module 622, conversion module 623, and padding module 625, which are included in the urine volume determination module. Since the content of specific operations is described above in the part describing the urine volume determination module, a redundant description will be omitted.

The segmentation module 1024 may segment a spectrogram converted by the conversion module 1023 by a predetermined time length. In this case, the segmentation module 1024 may segment the spectrogram so that the segmented spectrograms do not overlap each other. Without being limited thereto, the segmentation module 1024 may also segment a spectrogram so that some sections of the respective segmented spectrograms overlap with other adjacent segmented spectrograms.

A predetermined time length may be the same as the predetermined time length considered when the training data of the urination presence/absence determination model 1040 is processed. As an example, the predetermined time length may be a value between two seconds and ten seconds.

Meanwhile, in a case where the urination presence/absence determination model 1040 is a model trained to output urination presence/absence determination values by using sound data itself, the segmentation module 1024 may be configured to segment the sound data 1010 by a predetermined time length.

The urination presence/absence determination model 1040 may be a model trained to receive an input of data of a urination process and output data including a value for classifying a urination section or a non-urination section during the urination process. As an example, the urination presence/absence determination model 1040 may be a model trained to output urination presence/absence determination values by using a spectrogram, and may be a model trained in the above-described training method of the urination presence/absence determination model. Without being limited thereto, the urination presence/absence determination model 1040 may be a model trained to output urination presence/absence determination values by using sound data itself or other feature data.

Referring to FIG. 11, the urination presence/absence determination model 1040 may be configured to output first to n-th segmented urination presence/absence determination values 1050 corresponding to respective segmented spectrograms by using first to n-th segmented spectrograms 1030 segmented by the segmentation module 1024.

The concatenation module 1060 may be configured to output a urination presence/absence determination value 1070 corresponding to the sound data 1010 by concatenating the first to n-th segmented urination presence/absence determination values 1050 over time.

In a case where the segmentation module 1024 segments a spectrogram so that segmented spectrograms do not overlap each other, the concatenation module 1060 may output one urination presence/absence determination value 1070 by arranging the first to n-th segmented urination presence/absence determination values 1050 over time in a row.

In a case where the segmentation module 1024 segments a spectrogram so that segmented spectrograms partially overlap each other, the concatenation module 1060 arranges first to n-th segmented urination presence/absence determination values 1050 over time in a row. However, regarding the determination values in a section where these determination values overlap with adjacent segmented urination presence/absence determination values among the segmented urination presence/absence determination values, the concatenation module 1060 may determine, as a urination presence/absence determination value for the section, an average value or any other statistical value of the segmented urination presence/absence determination values belonging to the corresponding section.

As described above, the urination presence/absence determination module 1000 segments a spectrogram by a predetermined length, obtains segmented urination presence/absence determination values by using segmented spectrograms, and obtains a total urination presence/absence determination value by concatenating the segmented urination presence/absence determination values. Even though sound data of various lengths is input, the urination presence/absence determination model may determine urination presence/absence by using a spectrogram having a predetermined length, so there is an effect of reducing the influence of the total length of the sound data on the accuracy of a urination presence/absence determination result.

Meanwhile, the urination presence/absence determination module 1000 has been described as receiving the sound data 1010 and outputting the urination presence/absence determination value 1070, but it is not limited thereto. The urination presence/absence determination module may also be configured to receive an input of a spectrogram or other feature data itself and output a urination presence/absence determination value. In this case, the above-described crop module 622 may be configured to crop between urination start and end time points in a spectrogram, and the conversion module 623 may also not be included in the preprocessing module 620.

Meanwhile, the urination presence/absence determination module 1000 is not required to include all the filter module 1021, the crop module 1022, the conversion module 1023, the segmentation module 1024, and the padding module 1025. As an example, the urination presence/absence determination module 1000 may include merely some modules from among the filter module 1021, crop module 1022, conversion module 1023, segmentation module 1024, and padding module 1025, or may also not include the preprocessing module 1020 itself. In a case when the urination presence/absence determination module 1000 does not include the segmentation module 1024, the urination presence/absence determination module 1000 may also not include the concatenation module 1060. In a case where the urination presence/absence determination module 1000 does not include the preprocessing module 1020, the urination presence/absence determination module 1000 may also be configured such that the urination presence/absence determination model 1040 outputs urination presence/absence determination values by using the sound data 1010 itself.

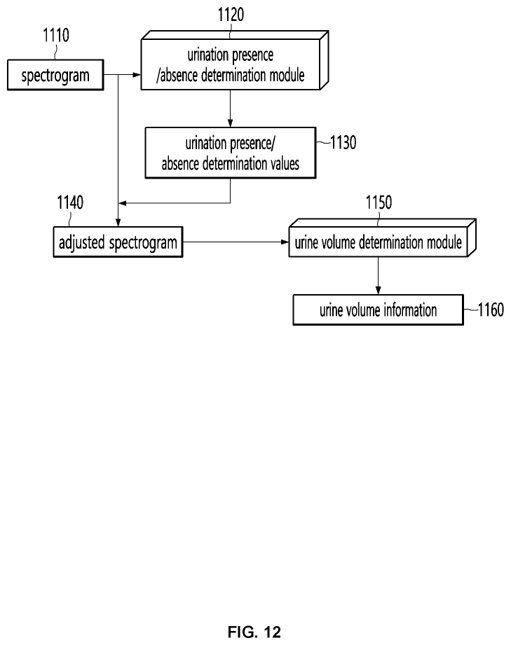

The urine volume determination module and urination presence/absence determination module may also be used together in order to obtain highly accurate urine volume information. Specific details will be described with reference to FIG. 12.

FIG. 12 is a view illustrating a method of obtaining urine volume information according to the exemplary embodiment.

Referring to FIG. 12, the sound analysis system may obtain urine volume information 1160 by using a urination presence/absence determination module 1120 and a urine volume determination module 1150 together. Specifically, the sound analysis system may obtain the urine volume information 1160 by using the urine volume determination module 1150 and an adjusted spectrogram 1140 to which an output value 1130 of the urination presence/absence determination module 1120 is applied to the spectrogram 1110.

In FIG. 12, the urination presence/absence determination module 1120 is configured to output urination a presence/absence determination value 1130 by using the spectrogram 1110, and the urine volume determination model 1150 is also configured to output the urine volume information 1160 by using the spectrogram 1140, but it is not limited thereto. The urination presence/absence determination module 1120 and/or the urine volume determination module 1150 may also be configured to output determination values by using sound data or other feature data.

As an example, in a case where the urination presence/absence determination module 1120 is configured to output a urination presence/absence determination value 1130 by using sound data and also the urine volume determination module 1150 is configured to output urine volume information 1150 by using a spectrogram, the sound analysis system may perform an operation of converting the sound data into the spectrogram at the same time, and may generate an adjusted spectrogram 1140 by applying the urination presence/absence determination value 1130 to the converted spectrogram. Since the details related to the type of data used by the urination presence/absence determination module 1120 and the urine volume determination module 1150 have been described above, a redundant description will be omitted.

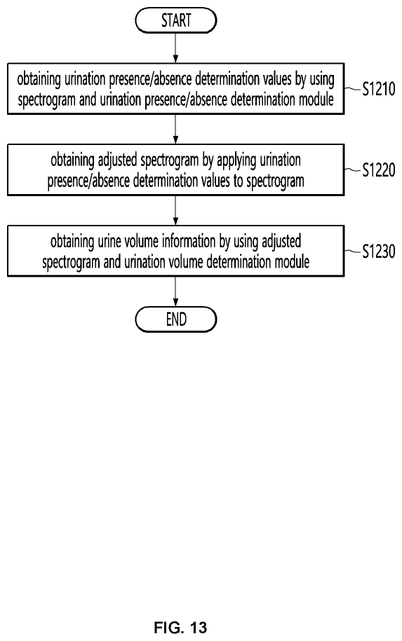

Specifically, a method of obtaining urine volume information will be described with reference to FIG. 13.

FIG. 13 is a flowchart illustrating a method of obtaining urine volume information according to the exemplary embodiment.

Referring to FIG. 13, in step S1210, the sound analysis system may obtain urination presence/absence determination values by using a spectrogram and a urination presence/absence determination module. The spectrogram may have been obtained by using sound data recorded during a urination process. Since the content related to using the spectrogram to output the urination presence/absence determination value by the urination presence/absence determination module has been described above, a redundant description will be omitted.

Meanwhile, in a case where the urination presence/absence determination module is configured to output a urination presence/absence determination value by using sound data, the sound analysis system may also obtain a urination presence/absence determination value by using the sound data and the urination presence/absence determination module.

Meanwhile, in a case where the urination presence/absence determination module is configured to output a urination presence/absence determination value by using feature data, the sound analysis system may also obtain a urination presence/absence determination value by using the feature data and the urination presence/absence determination module.

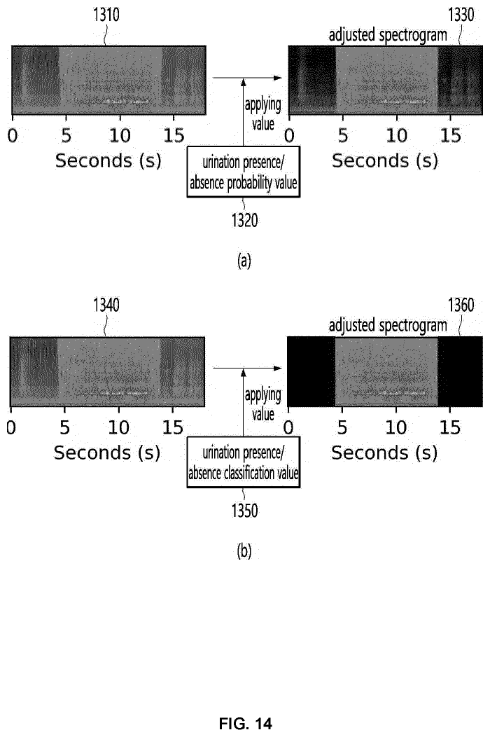

In step S1220, the sound analysis system may obtain an adjusted spectrogram by applying the urination presence/absence determination value to the spectrogram. As an example, the sound analysis system may obtain an adjusted spectrogram by performing a convolution on the urination presence/absence determination value and values of the spectrogram. As another example, the sound analysis system may also obtain an adjusted spectrogram by multiplying the urination presence/absence determination value by each value corresponding to the same time point of the spectrogram. Applying the urination presence/absence determination value to the spectrogram may be understood as reflecting the urination presence/absence determination value in the spectrogram. The content related to the adjusted spectrogram will be described below with reference to FIG. 14.

Meanwhile, in a case where the urine volume determination module is configured to output urine volume determination values by using sound data, the sound analysis system may also obtain adjusted sound data by applying a urination presence/absence determination value to the sound data. Alternatively, in a case where the urine volume determination module is configured to output urine volume determination values by using feature data, the sound analysis system may also obtain adjusted feature data by applying the urination presence/absence determination value to the feature data. Hereinafter, a component described as an adjusted spectrogram is intended to facilitate understanding of the content. Without further description, it is natural that even in a case where the component described as the adjusted spectrogram is implemented with the above-mentioned adjusted sound data or adjusted feature data, the case may be included in the technical idea of the present disclosure.

In step S1230, the sound analysis system may obtain urine volume information by using the adjusted spectrogram and a urine volume determination module. Since the adjusted spectrogram is considered with a urination presence/absence determination value, noise and the like other than sound related to a urination process may already be removed. Accordingly, the urine volume determination module outputs urine volume information by using the adjusted spectrogram without noise, so that the sound analysis system may obtain highly accurate urine volume information. Since the content related to using the spectrogram to output the urine volume determination value by the urine volume determination module has been described above, a redundant description will be omitted.

Meanwhile, in a case where the urine volume determination module is configured to output a urine volume determination value by using sound data, the sound analysis system may also obtain urine volume information by using the adjusted sound data and the urine volume determination module.

FIG. 14 is a view illustrating an adjusted spectrogram according to the exemplary embodiment.

Referring to FIG. 14(a), an adjusted spectrogram 1330 may be obtained by applying a urination presence/absence determination value to a spectrogram 1310. The applying of the urination presence/absence determination value may refer to a convolution on the urination presence/absence determination value and each value of the spectrogram. Without being limited thereto, the applying of the urination presence/absence determination value may refer to multiplying the urination presence/absence determination value by each value corresponding the same time point of the spectrogram.

As in FIG. 14(a), in a case where a urination presence/absence determination value is a urination presence/absence probability value 1320, a value close to 0 may be multiplied for a non-urination section of the spectrogram 1310, and a value close to 1 may be multiplied for a urination section of the spectrogram 1310. Accordingly, a difference between a value of the spectrogram 1310 in the non-urination section and a value of the adjusted spectrogram 1330 may be greater than a difference between a value of the spectrogram 1310 in the urination section and a value of the adjusted spectrogram 1330.

Referring to FIG. 14(b), an adjusted spectrogram 1360 may be obtained by applying a urination presence/absence determination value to a spectrogram 1340. The applying of the urination presence/absence determination value may refer to a convolution on the urination presence/absence determination value and each value of the spectrogram. Without being limited thereto, the applying of the urination presence/absence determination value may refer to multiplying the urination presence/absence determination value by each value corresponding the same time point of the spectrogram.

As in FIG. 14(b), in a case where a urination presence/absence determination value is a urination presence/absence classification value 1350, a value close to 0 may be multiplied for a non-urination section of the spectrogram 1340, and a value close to 1 may be multiplied for a urination section. Accordingly, the values of the adjusted spectrogram 1360 in the non-urination section may be zero, and the values of the adjusted spectrogram 1360 in the urination section may be the same as the values of the corresponding section in the spectrogram 1340.

Meanwhile, FIG. 14 shows that the adjusted spectrogram is obtained by applying the urination presence/absence determination value to the spectrogram, but obtaining adjusted sound data by applying the urination presence/absence determination value to the sound data may be similarly performed.

5. Method of Obtaining Urination Information by Using Urine Flow Rate Determination Model

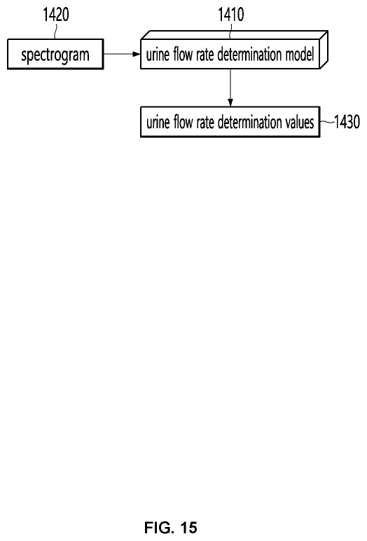

FIG. 15 is a view illustrating a urine flow rate determination model 1410 according to the exemplary embodiment.

The urine flow rate determination model 1410 may be a model trained to receive an input of data of a urination process and output urine flow rate data. The urine flow rate data may include values regarding urine flow rates during a urination process. The urine flow rate data may be understood as vector data, matrix data, or data in other formats.

Referring to FIG. 15, the data of a urination process may be a spectrogram 1420, and the urine flow rate data may be urine flow rate determination values 1430. The urine flow rate determination model 1410 may output the urine flow rate determination values 1430 over time predicted during the urination process corresponding to the input spectrogram 1420.

The urine flow rate determination values 1430 may be values reflecting change in the urine flow rates over time during the urination process corresponding to the spectrogram 1420.

The urine flow rate determination model 1410 may refer to a model trained by using machine learning. Here, the machine learning may be understood as a comprehensive concept that includes an artificial neural network and further includes deep learning. As an algorithm thereof, at least any one of k-nearest neighbors, linear regression, logistic regression, a support vector machine, a decision tree, a random forest, or a neural network may be used. Here, at least one of ANN, TDNN, DNN, CNN, RNN, or LSTM may be selected as the neural network.

Since the content related to the spectrogram used by the urine flow rate determination model 1410 has been described above in the part describing the urine volume determination model, a redundant description will be omitted.

Meanwhile, the urine flow rate determination model 1410 may also output urine flow rate determination values by using sound data itself or other feature data obtained from the sound data. Since the content related to the feature data has been described above, a redundant description will be omitted.

The urine flow rate determination model may be trained by using training data, and the training data may include sound data of a urination process and urine flow rate values of the urination process.

Since the content related to the sound data of the urination process has been described above in the part describing the urine volume determination model, a redundant description will be omitted.

A urine flow rate value of a urination process may be a measured urine flow rate value measured during the urination process corresponding to sound data.

In addition, for the purpose of improving training data, the urine flow rate values of the urination process (i.e., the training data) may include virtual urine flow rate values derived from the measured urine flow rate values. Hereinafter, a component described as measured urine flow rate values may be understood as including urine flow rate values derived from the measured urine flow rate values.

As an example, measured urine flow rate values may be obtained on the basis of data on toilet weight changes obtained during a urination process. Specifically, after setting two pieces of data so that a time domain of sound data obtained during the urination process and a time domain of data on a toilet weight change are the same as each other, a toilet weight change value in a section in which a toilet weight change is detected may be assigned as a urine flow rate value to the sound data. Alternatively, a value obtained by multiplying the toilet weight change value by a predetermined coefficient may also be assigned as a urine flow rate value to the sound data.

As another example, measured urine flow rate values may be obtained on the basis of data on collected urine volume changes obtained during a urination process. Specifically, after setting two pieces of data so that a time domain of sound data obtained during the urination process and a time domain of data on the collected urine volume changes are the same as each other, a collected urine volume change value in a section in which the collected urine volume change is detected may be assigned as a urine flow rate value to the sound data.

Without being limited thereto, various methods may be used to obtain a urine flow rate value during a urination process.

Meanwhile, the urine flow rate determination model may also be trained by using training data on which separate preprocessing has been performed.

As preprocessing, filtering for removing noise may be performed on sound data. Since the filtering has been described above in the part describing the urine volume determination model, a redundant description will be omitted.

As preprocessing, cropping of a section corresponding to a urination section may be performed in sound data. Since the cropping of the section corresponding to the urination section has been described in the part describing the urine volume determination model, a redundant description will be omitted.

As preprocessing, a task of converting sound data into separate feature data may be performed. As an example, feature data may be a spectrogram, and since the converting into the feature data has been described above in the part describing the urine volume determination model, a redundant description will be omitted.

As preprocessing, a spectrogram may be segmented by a predetermined time length. Since segmenting a spectrogram has been described above in the part describing the urination presence/absence determination model, a redundant description will be omitted.

As preprocessing, measured urine flow rate values may also be segmented by a predetermined time length, so as to correspond to a time section where a spectrogram is segmented.

The segmented measured urine flow rate values corresponding to the segmented spectrograms may have consecutive values (e.g., 0, 5, 11, 13, 12, 7, 3, 0, etc.) over time while in the segmented sections. Values in a specific time section and included in the segmented measured urine flow rate values may correspond to values in the same time section in the corresponding segmented spectrograms.

In a case when each of the total time lengths for a spectrogram and measured urine flow rate values is not an integer multiple of a predetermined time length, each length of a last segmented spectrogram and a last segmented measured urine flow rate value may be shorter than the predetermined time length. In this case, as preprocessing, padding may be further performed on the last segmented spectrogram and the last segmented measured urine flow rate value. Since the padding has been described above in the part describing the urination presence/absence determination model, a redundant description will be omitted.

Meanwhile, preprocessing for the above-described training data is not essential, and some of the above-described preprocessing processes may be omitted or the preprocessing itself may also not be performed. In a case when the task of converting sound data into separate feature data as preprocessing is omitted, the segmenting by the predetermined time length and the padding may also be performed on sound data itself as preprocessing.

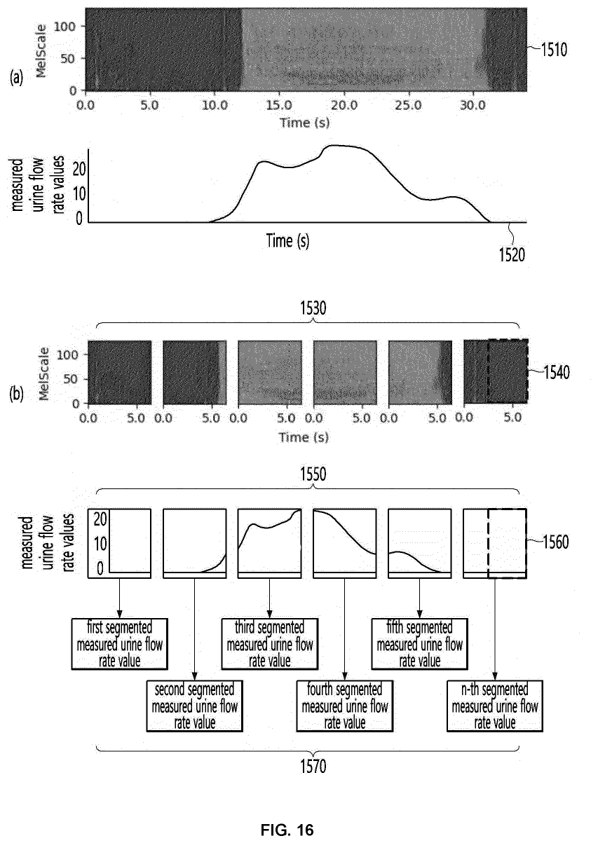

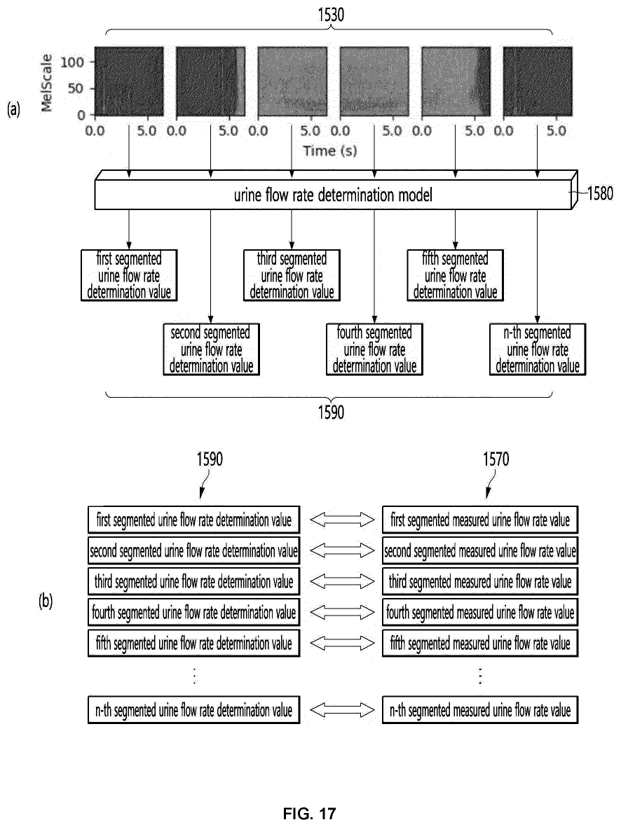

Specifically, a training process of a urine flow rate determination model will be described with reference to FIGS. 16 and 17.

FIGS. 16 and 17 are views illustrating a training process of a urine flow rate determination model 1580 according to the exemplary embodiment.

FIG. 16(a) shows a spectrogram 1510 converted from sound data recorded during a urination process and measured urine flow rate values 1520 corresponding to the sound data.

FIG. 16(b) shows segmented spectrograms 1530 obtained by segmenting the spectrogram 1510 by a predetermined time length of 6.4 seconds, and segmented measured urine flow rate values 1550 obtained by segmenting the measured urine flow rate values 1520 with the predetermined time length of 6.4 seconds.

FIG. 16 shows that the spectrogram 1510 is segmented into six segmented spectrograms and the measured urine flow rate value 1520 is segmented into six segmented measured urine flow rate values, but this is according to an example. The number of segmented spectrograms, the number of segmented measured urine flow rate values, and the predetermined time length are not limited thereto.

Referring to FIG. 16(b), each of time lengths of the first spectrogram 1510 and the measured urine flow rate values 1520 is not an integer multiple of the predetermined time length of 6.4 seconds, so a time length of a last segmented spectrogram and a time length of a last segmented measured urine flow rate value may be shorter than the predetermined time length. In this case, as described above, padding may be performed on the short time sections 1540 and 1560. As an example, zero padding for adding values of zeros may be performed. Consequently, it is possible to obtain segmented measured urine flow rate values 1570 having the same time lengths as each other and corresponding to respective segmented spectrograms 1530 having the same time lengths as each other. The segmented measured urine flow rate values 1570 may have consecutive values over time while in segmented time sections.

FIG. 17 (a) shows that a urine flow rate determination model 1580 uses the segmented spectrograms 1530 to output segmented urine flow rate determination values 1590 corresponding to the segmented spectrograms 1530. As an example, the urine flow rate determination model 1580 may output a first segmented urine flow rate determination value corresponding to a first segmented spectrogram, output a second segmented urine flow rate determination value corresponding to a second segmented spectrogram, . . . , and output an n-th segmented urine flow rate determination value corresponding to an n-th segmented spectrogram.

As in FIG. 17(b), the urine flow rate determination model 1580 may be trained so that the respective segmented urine flow rate determination values 1590 correspond to segmented measured urine flow rate values 1570.

The urine flow rate determination model 1580 may be trained through various methods such as supervised training, unsupervised training, reinforcement training, and imitation training. As an example, the urine flow rate determination model 1580 may be trained by comparing the segmented urine flow rate determination values 1590 with the segmented measured urine flow rate values 1570 and backpropagating an error thereof.

Meanwhile, it was described that the urine flow rate determination model 1580 is trained by using the segmented spectrograms 1530 obtained by segmenting the spectrogram 1510 and the segmented measured urine flow rate values 1570 obtained by segmenting the measured urine flow rate values 1520, but it is not limited thereto. The urine flow rate determination model may be configured to output a urine flow rate determination value for the entire section by using the unsegmented spectrogram itself, and may also be configured to be trained by using a urine flow rate determination value for the entire output section and the measured urine flow rate value.

Meanwhile, the urine flow rate determination model has been described as outputting urine flow rate determination values by using a spectrogram, but it is not limited thereto, and the urine flow rate determination model may also be configured to output urine flow rate determination values by using other feature data or sound data itself other than the spectrogram. Since the feature data has been described above, a redundant description will be omitted.

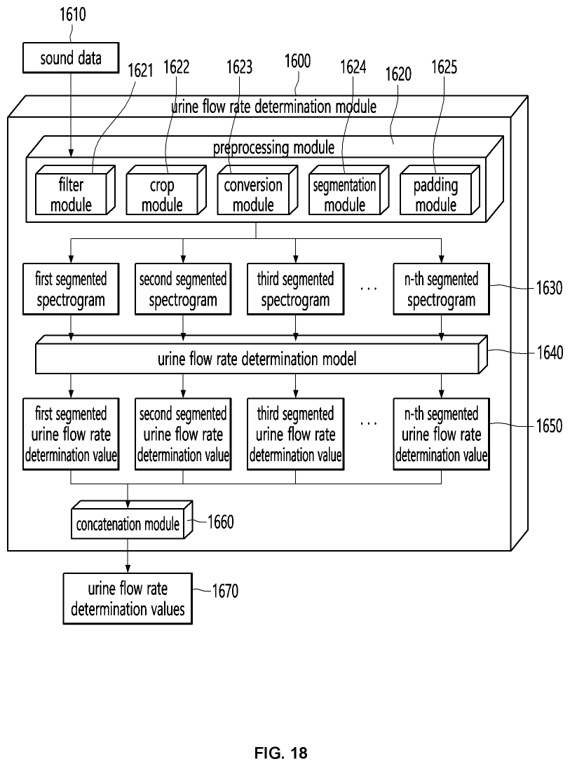

FIG. 18 is a view illustrating a configuration of a urine flow rate determination module 1600 according to the exemplary embodiment.

Referring to FIG. 18, the urine flow rate determination module 1600 may output urine flow rate determination values 1670 by using sound data 1610. Without being limited thereto, the urine flow rate determination module 1600 may also be configured to output a urine flow rate determination value by using a spectrogram or other feature data. The urine flow rate determination value may refer to urine flow rate data, and the urine flow rate data may include a value regarding a urine flow rate during a urination process.

The urine flow rate determination module 1600 according to one example may include a preprocessing module 1620, a urine flow rate determination model 1640, and a concatenation module 1660.

The preprocessing module 1620 may include a filter module 1621, a crop module 1622, a conversion module 1623, a segmentation module 1624, and/or a padding module 1625.

The filter module 1621, crop module 1622, conversion module 1623, and padding module 1625 may operate similarly to the filter module 621, crop module 622, conversion module 623, and padding module 625, which are included in the urine volume determination module 600 described above. Since the details of the operation are described in the part describing the urine volume determination module above, a redundant description will be omitted.

The segmentation module 1624 may operate similarly to the segmentation module 1024 included in the urination presence/absence determination module 1000, and since the details of the operation are described in the part describing the urination presence/absence determination module above, a redundant description will be omitted.

The urine flow rate determination model 1640 is a model trained to receive an input of data of a urination process and output data including urine flow rate values during a urination process. As an example, the urine flow rate determination model 1640 may be a model trained to output urine flow rate determination values by using a spectrogram, and may be a model trained with the above-described training method of the urine flow rate determination model. Without being limited thereto, the urine flow rate determination model 1640 may also be a model trained to output urine flow rate determination values by using sound data itself or other feature data.

Referring to FIG. 18, the urine flow rate determination model 1640 may be configured to output first to n-th segmented urine flow rate determination values 1650 corresponding to respective segmented spectrograms by using the first to n-th segmented spectrograms 1630 segmented by the segmentation module 1624.

The concatenation module 1660 may be configured to output the urine flow rate determination values 1670 corresponding to the sound data 1610 by concatenating the first to n-th segmented urine flow rate determination values 1650 over time.

The concatenation module 1660 may operate similarly to the concatenation module 1060 included in the urination presence/absence determination module 1000, and since the details of the operation are described in the part describing the urination presence/absence determination module above, a redundant description will be omitted.

As described above, the urine flow rate determination module 1600 segments a spectrogram by a predetermined length, obtains segmented urine flow rate determination values by using segmented spectrograms, and obtains a total urine flow rate determination value by concatenating the segmented urine flow rate determination values. Even though sound data of various lengths is input, the urine flow rate determination module may determine a urine flow rate by using the spectrogram of the predetermined length, so there is an effect of reducing the influence of the total length of the sound data on the accuracy of a urine flow rate determination result.

Meanwhile, the urine flow rate determination module 1600 has been described as outputting the urine flow rate determination values 1670 after receiving an input of the sound data 1610, but it is not limited thereto. The urine flow rate determination module may also be configured to receive an input of a spectrogram or other feature data itself and output a urine flow rate determination value. In this case, the conversion module 1623 may not be included in the preprocessing module 1620.

Meanwhile, the urine flow rate determination module 1600 is not required to include all the filter module 1621, crop module 1622, conversion module 1623, segmentation module 1624, and padding module 1625. As an example, the urine flow rate determination module 1600 may include merely some modules from among the filter module 1621, crop module 1622, conversion module 1623, segmentation module 1624, and padding module 1625, or may also not include the preprocessing module 1620 itself.

In a case when the urine flow rate determination module 1600 does not include the segmentation module 1624, the urine flow rate determination module 600 may also not include the concatenation module 1660. In a case where the urine flow rate determination module 1600 does not include the preprocessing module 1620, the urine flow rate determination module 1600 may be configured to allow the urine flow rate determination model 1640 to output urine flow rate determination values by using the sound data 1610 itself.

The urine flow rate determination module, the urination presence/absence determination module, and/or the urine volume determination module may also be used together in order to obtain highly accurate urine volume information. The details will be described below.

6. Method of Obtaining Urination Information by Using Urine Volume Determination Module, Urination Presence/Absence Determination Module, and Urine Flow Rate Determination Module

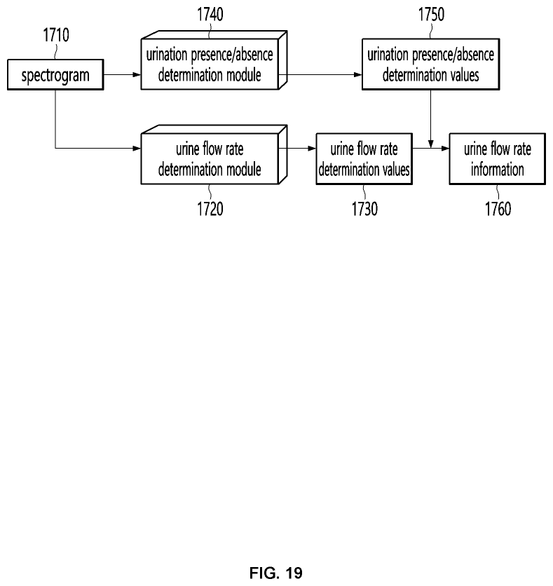

FIG. 19 is a view illustrating a method of obtaining urine flow rate information according to the exemplary embodiment.

Referring to FIG. 19, the sound analysis system may obtain urine flow rate information 1760 by using a urine flow rate determination module 1720 and a urination presence/absence determination module 1740.

As an example, the sound analysis system may obtain urine flow rate information 1760 by applying urination presence/absence determination values 1750 output by the urination presence/absence determination module 1740 to urine flow rate determination values 1730 output by the urine flow rate determination module 1720. Specifically, the sound analysis system may obtain the urine flow rate determination value 1730 by using the spectrogram 1710 and the urine flow rate determination module 1720. In addition, the sound analysis system may obtain urination presence/absence determination values 1750 by using the spectrogram 1710 and the urination presence/absence determination module 1740.

The spectrogram may have been obtained by using sound data recorded during a urination process. Since the content related to using the spectrogram to output the urine flow rate determination values and the urination presence/absence determination values respectively by the urine flow rate determination module and the urination presence/absence determination module has been described above, a redundant description will be omitted.

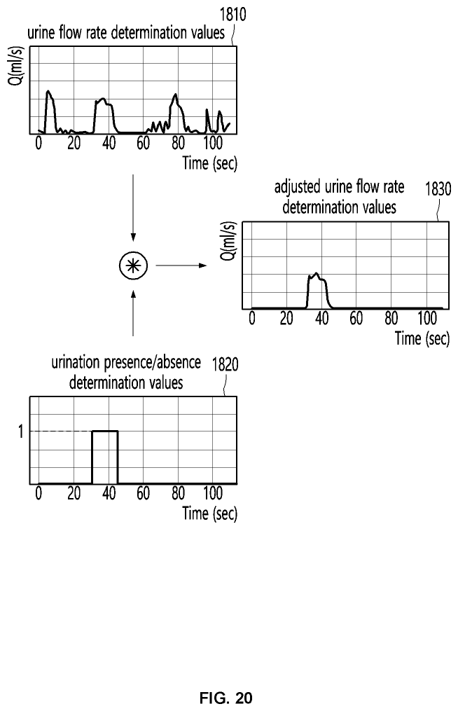

The sound analysis system may obtain urine flow rate information by applying urination presence/absence determination values to urine flow rate determination values. As an example, the sound analysis system may obtain urine flow rate information by performing a convolution on urination presence/absence determination values and urine flow rate determination values. As another example, the sound analysis system may obtain urine flow rate information by multiplying respective values with each other corresponding to identical time points of urination presence/absence determination values and urine flow rate determination values. Details related to applying the urination presence/absence determination values will be described with reference to FIG. 20.

FIG. 20 is a view illustrating applying urination presence/absence determination values according to the exemplary embodiment.

Referring to FIG. 20, adjusted urine flow rate determination values 1830 may be obtained by applying urination presence/absence determination values 1820 to urine flow rate determination values 1810. The applying of the urination presence/absence determination values 1820 may refer to a convolution on the urination presence/absence determination values 1820 and the urine flow rate determination values 1830. Without being limited thereto, the applying of the urination presence/absence determination values may refer to multiplying respective values with each other corresponding identical time points of the urination presence/absence determination values and the urine flow rate determination values. The applying of the urination presence/absence determination values may refer to adjusting the urine flow rate determination values while considering the urination presence/absence determination values, or may refer to reflecting the urination presence/absence determination values in the urine flow rate determination values.

Since the urination presence/absence determination values 1820 are considered in the adjusted urine flow rate determination values 1830, noise and the like other than sound related to a urination process may be removed from the adjusted urine flow rate determination values 1830. Accordingly, the sound analysis system may obtain highly accurate urine flow rate information.

Meanwhile, in FIG. 19, the urine flow rate determination module 1720 is configured to output the urine flow rate determination values 1730 by using the spectrogram 1710, and the urination presence/absence determination module 1740 is also configured to output the urination presence/absence determination values 1750 by using the spectrogram 1710, but it is not limited thereto. The urine flow rate determination module 1720 and/or the urination presence/absence determination module 1740 may also be configured to output determination values by using sound data or other feature data, and since the specific details thereof have been described above, a redundant description will be omitted.

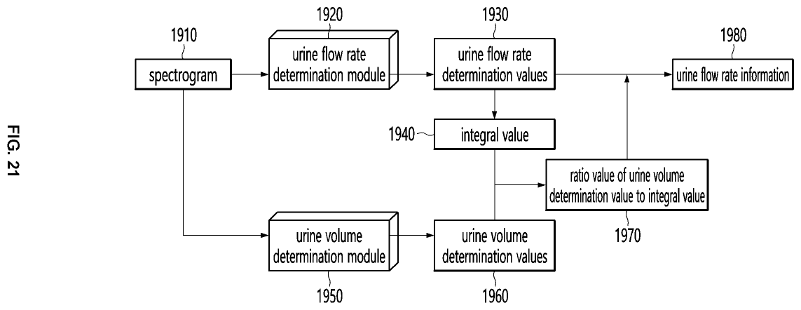

FIG. 21 is a view illustrating a method of obtaining urine flow rate information according to the exemplary embodiment.

Referring to FIG. 21, the sound analysis system may obtain urine flow rate information 1980 by using a urine flow rate determination module 1920 and a urine volume determination module 1950. Specifically, the sound analysis system may obtain the urine flow rate information 1980 by applying urine volume determination values 1960 output by the urine volume determination module 1950 to urine flow rate determination values 1930 output by the urine flow rate determination module 1920.

More specifically, the sound analysis system may obtain the urine flow rate determination values 1930 by using a spectrogram 1910 and the urine flow rate determination module 1920. In addition, the sound analysis system may obtain urine volume determination values 1960 by using the spectrogram 1910 and the urine volume determination module 1950.

The spectrogram may have been obtained by using sound data recorded during a urination process. Since the content related to using the spectrogram to output the urine flow rate determination values and the urine volume determination values respectively by the urine flow rate determination module and the urine volume determination module has been described above, a redundant description will be omitted.

In FIG. 21, the urine flow rate determination module 1920 is configured to output the urine flow rate determination values 1930 by using the spectrogram 1910, and the urine volume determination module 1950 is also configured to output the urine volume determination values 1960 by using the spectrogram 1910, but it is not limited thereto. The urine flow rate determination module 1920 and/or the urine volume determination module 1950 may also be configured to output determination values by using sound data or other feature data, and since the specific details thereof have been described above, a redundant description will be omitted.

The sound analysis system may obtain an integral value 1940 by integrating the urine flow rate determination values 1930 over time. Since a urine flow rate refers to a urine volume per unit time, the total urine volume may be obtained as the integral value 1940 in a case where the urine flow rate determination values 1930 are integrated over time.

The sound analysis system may calculate a ratio value 1970 of the urine volume determination values 1960 to the obtained integral value 1940. A difference between a urine volume value obtained from the urine flow rate determination module 1920 and a urine volume determination value obtained from urine volume determination module 1950 may be reflected in the ratio value 1970.

The sound analysis system may obtain urine flow rate information 1980 by applying the calculated ratio value 1970 to the urine flow rate determination values 1930. The applying of the ratio value 1970 to the urine flow rate determination values 1930 may refer to multiplying each of the urine flow rate determination values 1930 over time by the ratio value 1970. Without being limited thereto, the applying of the ratio value 1970 to each of the urine flow rate determination values 1930 may refer to adjusting each of the urine flow rate determination values 1930 by using the ratio value 1970, and refer to reflecting the ratio value 1970 in each of the urine flow rate determination values 1930.

Since the accuracy of each urine volume determination value obtained by using the urine volume determination module 1950 may be higher than that of a urine flow value obtained by integrating the urine flow rate determination values 1930 over time, more accurate urine flow rate information 1980 may be obtained in a case where the ratio value 1970 obtained by using the urine volume determination values 1960 applied to a urine flow rate waveform of the urine flow rate determination values 1930.

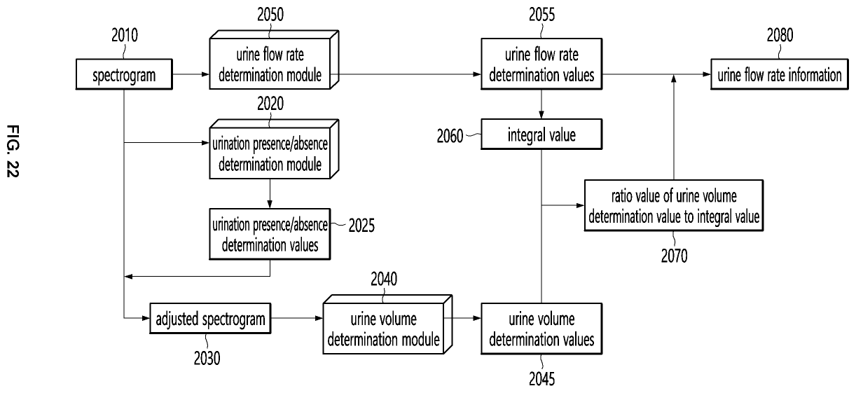

Meanwhile, the sound analysis system may obtain urine flow rate information by using all the urine flow rate determination module, urination presence/absence determination module, and urine volume determination module. Specific details will be described with reference to FIG. 22.

FIG. 22 is a view illustrating a method of obtaining urine flow rate information according to the exemplary embodiment.

Referring to FIG. 22, the sound analysis system may obtain urine flow rate information 2080 by using a urination presence/absence determination module 2020, a urine volume determination module 2040, and a urine flow rate determination module 2050. Specifically, the sound analysis system may obtain urine volume determination values 2045 by using the urination presence/absence determination module 2020 and the urine volume determination module 2040, and obtain the urine flow rate information 2080 by using urine flow rate determination values 2055 output by the urine flow rate determination module 2050 and urine volume determination values 2045.

More specifically, the sound analysis system may obtain urination presence/absence determination values 2025 by using a spectrogram 2010 and the urination presence/absence determination module 2020. The sound analysis system may obtain an adjusted spectrogram 2030 by applying the obtained urination presence/absence determination values 2025 to the spectrogram 2010. In addition, the sound analysis system may obtain urine volume determination values 2045 by using the adjusted spectrogram 2030 and the urine volume determination module 2040.

The spectrogram may have been obtained by using sound data recorded during a urination process. Since the content related to the sound analysis system obtaining the urine volume determination values by using the urination presence/absence determination module and the urine volume determination module together has been described in detail in FIGS. 12 and 13, a redundant description will be omitted.

In FIG. 22, a urination presence/absence determination module 2020 is configured to output the urination presence/absence determination values 2025 by using the spectrogram 2010, and the urine volume determination module 2040 is also configured to output the urine volume determination values 2045 by using the spectrogram 2030, but it is not limited thereto. The urination presence/absence determination module 2020 and/or the urine volume determination module 2040 may also be configured to output determination values by using sound data or other feature data, and since the specific details thereof have been described above, a redundant description will be omitted.

The sound analysis system may obtain the urine flow rate determination values 2055 by using the spectrogram 2010 and the urine flow rate determination module 2050. The spectrogram may have been obtained by using sound data recorded during the urination process. Since the content related to obtaining the urine flow rate determination values by using the spectrogram and the urine flow rate determination module have been described above, a redundant description will be omitted.

FIG. 22 shows that the urine flow rate determination module 2050 is configured to output the urine flow rate determination values 2055 by using the spectrogram 2010, but is not limited thereto. The urine flow rate determination module 2050 may also be configured to output urine flow rate determination values by using sound data or other feature data, and since the specific details thereof have been described above, a redundant description will be omitted.

The sound analysis system may obtain urine flow rate information 2080 by using the obtained urine flow rate determination values 2055 and urine volume determination values 2045. Specifically, the sound analysis system may obtain an integral value 2060 by integrating the urine flow rate determination values 2055 over time, and calculate a ratio value 2070 of the urine volume determination values 2045 with respect to the integral value 2060. Since the content related to the integral value and ratio value have been described above in FIG. 21, a redundant description will be omitted.

The sound analysis system may obtain more accurate urine flow rate information 2080 by applying the calculated ratio value 2070 to the urine flow rate determination values 2055. Since the content related to applying the ratio value 2070 to the urine flow rate determination values 2055 has been described in detail in FIG. 21, a redundant description will be omitted.

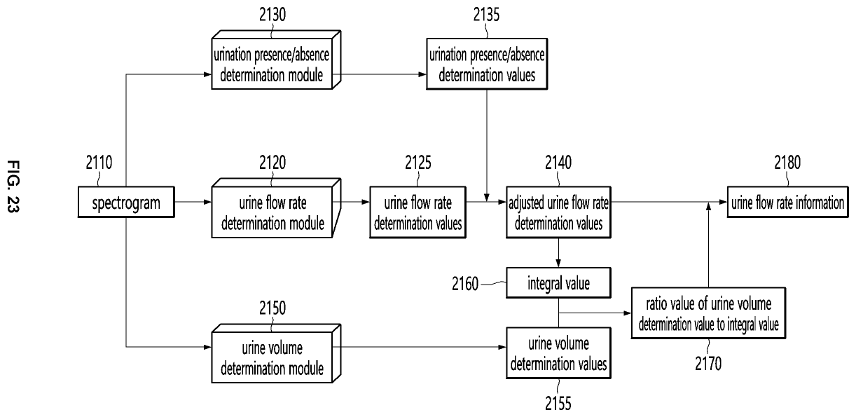

Meanwhile, the sound analysis system may also obtain urine flow rate information by arranging the urination presence/absence determination module in a different manner. Specific details will be described with reference to FIG. 23.

FIG. 23 is a view illustrating a method of obtaining urine flow rate information according to the exemplary embodiment.

Referring to FIG. 23, the sound analysis system may obtain urine flow rate information 2180 by using a urine flow rate determination module 2120, a urination presence/absence determination module 2130, and a urine volume determination module 2150. Specifically, the sound analysis system may obtain adjusted urine flow rate determination values 2140 by applying a urination presence/absence determination values 2135 output by the urination presence/absence determination module 2130 to urine flow rate determination values 2125 output by the urine flow rate determination module 2120. The sound analysis system may obtain the urine flow rate information 2180 by using urine volume determination values 2155, which is output by the urine volume determination module 2150, and adjusted urine flow rate determination values 2140.

More specifically, the sound analysis system may obtain the urine flow rate determination values 2125 by using a spectrogram 2110 and the urine flow rate determination module 2120. In addition, the sound analysis system may obtain the urination presence/absence determination values 2135 by using the spectrogram 2110 and the urination presence/absence determination module 2130.

The spectrogram may have been obtained by using sound data recorded during a urination process. Since the content related to using the spectrogram to output the urine flow rate determination values and the urination presence/absence determination values respectively by the urine flow rate determination module and the urination presence/absence determination module has been described above, a redundant description will be omitted.

In FIG. 23, the urine flow rate determination module 2120 is configured to output the urine flow rate determination values 2125 by using the spectrogram 2110, and the urination presence/absence determination module 2130 is also configured to output the urination presence/absence determination values 2135 by using the spectrogram 2110, but it is not limited thereto. The urine flow rate determination module 2120 and/or the urination presence/absence determination module 2130 may also be configured to output determination values by using sound data or other feature data, and since the specific details thereof have been described above, a redundant description will be omitted.

The sound analysis system may obtain adjusted urine flow rate determination values 2140 by applying the urination presence/absence determination values 2135 to the urine flow rate determination values 2125. Since the content related to applying the urination presence/absence determination values to the urine flow rate determination values have been described in detail in FIGS. 19 and 20, a redundant description will be omitted.

The sound analysis system may obtain the urine volume determination values 2155 by using the spectrogram 2110 and the urine volume determination module 2150.

The spectrogram may have been obtained by using sound data recorded during a urination process. Since the content related to the urine volume determination module using the spectrogram to output the urine volume determination values has been described above, a redundant description will be omitted.

FIG. 23 shows that the urine volume determination module 2150 is configured to output the urine volume determination values 2155 by using the spectrogram 2110, but it is not limited thereto. The urine volume determination module 2150 may also be configured to output determination values by using sound data or other feature data, and since the specific details thereof have been described above, a redundant description will be omitted.

The sound analysis system may obtain urine flow rate information 2180 by using the adjusted urine flow rate determination values 2140 and the urine volume determination values 2155. Specifically, the sound analysis system may obtain an integral value 2160 by integrating the adjusted urine flow rate determination values 2140 over time, and calculate a ratio value 2170 of the urine volume determination values 2155 to the integral value 2160. Since the content related to the integral value and ratio value have been described above in FIG. 21, a redundant description will be omitted.

The sound analysis system may obtain more accurate urine flow rate information 2180 by applying the calculated ratio value 2170 to the adjusted urine flow rate determination values 2140. Since the content related to applying the ratio value to the urine flow rate determination values has been described in detail in FIG. 21, a redundant description will be omitted.

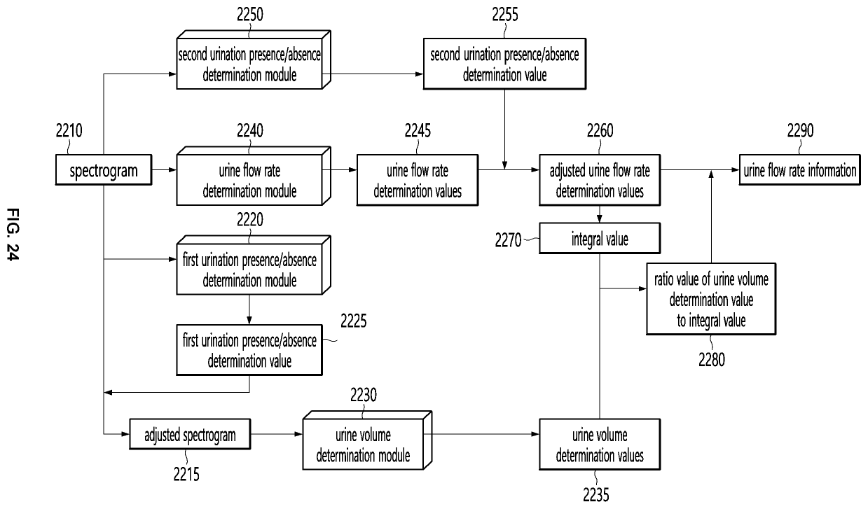

Meanwhile, in obtaining urine flow rate determination values and urine volume determination values to obtain urine flow rate information, the sound analysis system may use the urine flow rate determination module and the urination presence/absence determination module to obtain the urine flow rate determination values, and may also use the urine volume determination module and the urination presence/absence determination module to obtain the urine volume determination values. Specific details will be described with reference to FIG. 24.

FIG. 24 is a view illustrating a method of obtaining urine flow rate information according to the exemplary embodiment.

Referring to FIG. 24, the sound analysis system may obtain urine volume determination values 2235 by using a first urination presence/absence determination module 2220 and a urine volume determination module 2230, obtain adjusted urine flow rate determination values 2260 by using a second urination presence/absence determination module 2250 and a urine flow rate determination module 2240, and obtain urine flow rate information 2290 by using the adjusted urine flow rate determination values 2260 and the urine volume determination values 2235, which are obtained.

The first urination presence/absence determination module 2220 and the second urination presence/absence determination module 2250 may be configured with the same urination presence/absence determination modules as each other, but is not limited thereto. The first urination presence/absence determination module 2220 and the second urination presence/absence determination module 2250 may also be configured with urination presence/absence determination modules different from each other. As an example, the first urination presence/absence determination module 2220 may be a model trained to output a probability value for urination presence/absence, and the second urination presence/absence determination module 2250 may be a model trained to output a classification value for classifying the urination presence/absence, and vice versa.

The sound analysis system may obtain a first urination presence/absence determination value 2225 by using a spectrogram 2210 and the first urination presence/absence determination module 2220. The sound analysis system may obtain an adjusted spectrogram 2215 by applying the obtained first urination presence/absence determination value 2225 to the spectrogram 2010. In addition, the sound analysis system may obtain urine volume determination values 2235 by using the adjusted spectrogram 2215 and the urine volume determination module 2230.

The spectrogram may have been obtained by using sound data recorded during a urination process. Since the content related to the sound analysis system obtaining the urine volume determination values by using the urination presence/absence determination module and the urine volume determination module together has been described in detail in FIGS. 12 and 13, a redundant description will be omitted.

In FIG. 24, the first urination presence/absence determination module 2220 is configured to output a first urination presence/absence determination value 2225 by using the spectrogram 2210, and the urine volume determination module 2230 is also configured to output the urine volume determination values 2235 by using the spectrogram 2215, but it is not limited thereto. The first urination presence/absence determination module 2220 and/or the urine volume determination module 2230 may also be configured to output determination values by using sound data or other feature data, and since the specific details thereof have been described above, a redundant description will be omitted.

The sound analysis system may obtain urine flow rate determination values 2245 by using the spectrogram 2210 and the urine flow rate determination module 2240. In addition, the sound analysis system may obtain a second urination presence/absence determination value 2255 by using the spectrogram 2210 and the second urination presence/absence determination module 2250. The second urination presence/absence determination value 2255 may be the same as the first urination presence/absence determination value 2225, but may also have a different value.

The spectrogram may have been obtained by using sound data recorded during a urination process. Since the content related to the sound analysis system obtaining the adjusted urine flow rate determination values 2260 by using the urine flow rate determination module 2240 and the urination presence/absence determination module 2250 together has been described in detail in FIGS. 19 and 20, a redundant description will be omitted.

In FIG. 24, the urine flow rate determination module 2240 is configured to output the urine flow rate determination values 2245 by using the spectrogram 2210, and the second urination presence/absence determination module 2250 is also configured to output the second urination presence/absence determination value 2255 by using the spectrogram 2210, but it is not limited thereto. The urine flow rate determination module 2240 and/or the second urination presence/absence determination module 2250 may also be configured to output determination values by using sound data or other feature data, and since the specific details thereof have been described above, a redundant description will be omitted.

The sound analysis system may obtain urine flow rate information 2290 by using the obtained adjusted urine flow rate determination values 2260 and urine volume determination values 2235. Specifically, the sound analysis system may obtain an integral value 2270 by integrating the adjusted urine flow rate determination values 2260 over time, and calculate a ratio value 2280 of the urine volume determination values 2235 to the integral value 2270. Since the content related to the integral value and ratio value have been described above in FIG. 21, a redundant description will be omitted.

The sound analysis system may obtain more accurate urine flow rate information 2290 by applying the calculated ratio value 2280 to the adjusted urine flow rate determination values 2260. The applying of the ratio value 2280 to the adjusted urine flow rate determination values 2260 may refer to multiplying each of the adjusted urine flow rate determination values 2260 over time by the ratio value 2280. Without being limited thereto, the applying of the ratio value 2280 to the adjusted urine flow rate determination values 2260 may include adjusting the adjusted urine flow rate determination values 2260 by using the ratio value 2280.

Since the accuracy of the urine volume determination values 2235 obtained by using the first urination presence/absence determination module 2220 and the urine volume determination module 2230 may be higher than that of the urine flow rate determination value obtained by integrating the adjusted urine flow rate determination values 2260 over time, highly accurate urine flow rate information 2290 may be obtained in a case where the ratio value 2280 obtained by using the urine volume determination values 2235 is applied to a urine flow rate waveform of the adjusted urine flow rate determination values 2260.

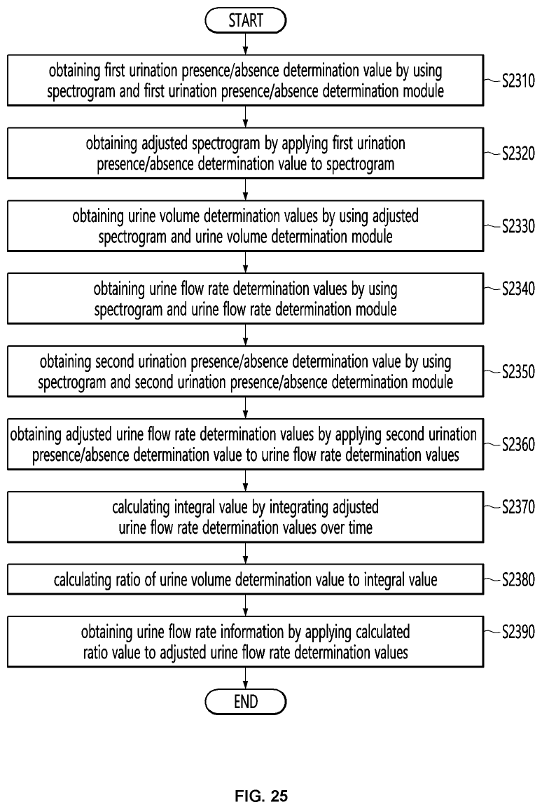

FIG. 25 is a flowchart illustrating the method of obtaining the urine flow rate information according to the exemplary embodiment.

Referring to FIG. 25, in step S2310, the sound analysis system may obtain a first urination presence/absence determination value by using a spectrogram and a first urination presence/absence determination module. The spectrogram may have been obtained by using sound data recorded during a urination process. Since the content related to using the spectrogram to output the urination presence/absence determination values by the urination presence/absence determination module has been described above, a redundant description will be omitted.

Meanwhile, in a case where the urination presence/absence determination module is trained to output urination presence/absence determination values by using sound data, the sound analysis system may also obtain urination presence/absence determination values by using the sound data and the urination presence/absence determination module.

In step S2320, the sound analysis system may obtain an adjusted spectrogram by applying the first urination presence/absence determination value to the spectrogram. Since the content related to obtaining the adjusted spectrogram have been described above, a redundant description will be omitted.

In step S2330, the sound analysis system may obtain urine volume determination values by using the adjusted spectrogram and a urine volume determination module. Since the content related to using the spectrogram to output the urine volume determination values by the urine volume determination module has been described above, a redundant description will be omitted.

In step S2340, the sound analysis system may obtain urine flow rate determination values by using the spectrogram and a urine flow rate determination module. Since the content related to using the spectrogram to output the urine flow rate determination values by the urine flow rate determination module has been described above, a redundant description will be omitted.

Meanwhile, in a case where the urine flow rate determination module is trained to output urine flow rate determination values by using sound data, the sound analysis system may also obtain urine flow rate determination values by using the sound data and the urine flow rate determination module.

In step S2350, the sound analysis system may obtain a second urination presence/absence determination value by using the spectrogram and a second urination presence/absence determination module. The second urination presence/absence determination module may be the same urination presence/absence determination module as the first urination presence/absence determination module, but is not limited thereto. The first urination presence/absence determination module and the second urination presence/absence determination module may also be configured with urination presence/absence determination modules different from each other, and since the specific contents have been described above, a redundant description will be omitted.

In step S2360, the sound analysis system may obtain adjusted urine flow rate determination values by applying the second urination presence/absence determination value to the urine flow rate determination values. Since the content related to obtaining the adjusted urine flow rate determination values by using the urination presence/absence determination values and the urine flow rate determination values have been described above, a redundant description will be omitted.

In step S2370, the sound analysis system may calculate an integral value by integrating the adjusted urine flow rate determination values over time. In step S2380, the sound analysis system may calculate a ratio of a urine volume determination value to the integral value. Since the content related to the integral value and ratio value have been described above, a redundant description will be omitted.

In step S2390, the sound analysis system may obtain urine flow rate information by applying the calculated ratio value to the adjusted urine flow rate determination values. Since the content related to applying the ratio value to the adjusted urine flow rate determination values has been described in detail, a redundant description will be omitted.

7. Method of Obtaining Urination Information by Using Urine Volume Determination Module, Urination Presence/Absence Determination Module, and Relative Urine Flow Rate Determination Module

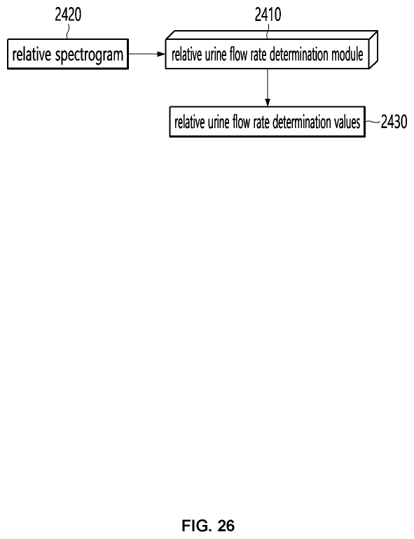

FIG. 26 is a view illustrating a relative urine flow rate determination model according to the exemplary embodiment.

A relative urine flow rate determination model 2410 may be a model trained to receive an input of data of a urination process and output relative urine flow rate data. The relative urine flow rate data may include normalized values regarding urine flow rates during a urination process. The relative urine flow rate data may be understood as vector data, matrix data, or data in other formats.

Referring to FIG. 26, data of a urination process may be a relative spectrogram 2420, and relative urine flow rate data may be relative urine flow rate determination values 2430. The relative urine flow rate determination model 2410 may output relative urine flow rate determination values 2430 over time predicted during the urination process corresponding to the input relative spectrogram 2420.

The relative spectrogram 2420 is the spectrogram in which all values are divided by the maximum value among values in a spectrogram. That is, the values of the relative spectrogram 2420 may be normalized values.

Meanwhile, a component described as a spectrogram in the present disclosure may be implemented with sound data itself or other feature data for the sound data. That is, the relative spectrogram 2420 may be composed of relative sound data or relative feature data. The relative sound data or relative feature data may be obtained from sound data or feature data in a method similar to the method of obtaining the relative spectrogram from the spectrogram.

The relative urine flow rate determination values 2430 may be values reflecting urine flow rate change over time during a urination process corresponding to the relative spectrogram 2420.

Having values between 0 and 1, the relative urine flow rate determination values 2430 provide a waveform for the urine flow rate change, and the relative urine flow rate determination values 2430 may be determined so that a maximum relative urine flow rate determination value corresponding to a maximum urine flow rate value becomes 1.

The relative urine flow rate determination model 2410 outputs the relative urine flow rate determination values 2430 by using the relative spectrogram 2420, so there is an effect of reducing the influence of absolute magnitudes of sound on determination values. Meanwhile, the same effect may be achieved even in a case where the relative urine flow rate determination model outputs relative urine flow rate determination values by using relative sound data or relative feature data.

The relative urine flow rate determination model 2410 may refer to a model trained by using machine learning. Here, the machine learning may be understood as a comprehensive concept that includes an artificial neural network and further includes deep learning. As an algorithm thereof, at least any one of k-nearest neighbors, linear regression, logistic regression, a support vector machine, a decision tree, a random forest, or a neural network may be used. Here, at least one of ANN, TDNN, DNN, CNN, RNN, or LSTM may be selected as the neural network.

The relative urine flow rate determination model may be trained by using training data, and the training data may include sound data of a urination process and urine flow rate values of the urination process.

Since the content related to the sound data of the urination process has been described above in the part describing the urine volume determination model, a redundant description will be omitted.

Urine flow rate values of a urination process may be measured urine flow rate values measured during the urination process corresponding to sound data. Since the content related to the measured urine flow rate values has been described above in the part describing the urine flow rate determination model, a redundant description will be omitted.

Meanwhile, the relative urine flow rate determination model may also be trained by using training data on which separate preprocessing has been performed.

As preprocessing, filtering for removing noise may be performed on sound data. Since the filtering has been described above in the part describing the urine volume determination model, a redundant description will be omitted.

As preprocessing, cropping of a section corresponding to a urination section may be performed in sound data. Since the cropping of the section corresponding to the urination section has been described in the part describing the urine volume determination model, a redundant description will be omitted.

As preprocessing, a task of converting sound data into separate feature data may be performed. As an example, feature data may be a spectrogram, and since converting into the feature data has been described above in the part describing the urine volume determination model, a redundant description will be omitted.

As preprocessing, a spectrogram may be converted into a relative spectrogram by dividing all values by the maximum value among the values of the spectrogram. In this way, the values of the spectrogram may be normalized. It is not limited thereto, and in a case where sound data is converted into other feature data as preprocessing, the values of the other feature data may also be normalized and converted into relative feature data. Meanwhile, in a case where the preprocessing to convert into feature data is not performed, the values of the sound data may also be normalized and converted into relative sound data.

As preprocessing, measured urine flow rate values may be converted into measured relative urine flow rate values by dividing all values by the maximum value among the measured urine flow rate values. In this way, the measured urine flow rate values may be normalized, and the measured relative urine flow rate values may have a waveform of the measured urine flow rate values.

As preprocessing, a relative spectrogram may be segmented by a predetermined time length. Since the segmenting of the relative spectrogram has been described above in the part describing the urination presence/absence determination model, a redundant description will be omitted.

As preprocessing, measured relative urine flow rate values may be segmented by a predetermined time length. Since the segmenting of the measured relative urine flow rate values may be performed similarly to the segmenting of the measured urine flow rate values described above in the part describing the urine flow rate determination model, a redundant description will be omitted.

In a case when each of the total time lengths for a relative spectrogram and measured relative urine flow rate values is not an integer multiple of a predetermined time length, each of lengths of a last segmented relative spectrogram and a last segmented measured relative urine flow rate value may be shorter than the predetermined time length.

In this case, as preprocessing, padding may be further performed on the last segmented relative spectrogram and the last segmented measured relative urine flow rate value. Since the padding has been described above in the part describing the urination presence/absence determination model, a redundant description will be omitted.

Meanwhile, the filtering, cropping, converting into feature data, segment by a predetermined time length, and/or padding of the preprocessing of training data described above are not essential, and some or all of them may also be omitted.

In a case when the task of converting sound data into separate feature data as preprocessing is omitted, the segmenting by the predetermined time length and the padding may also be performed on sound data itself as preprocessing.

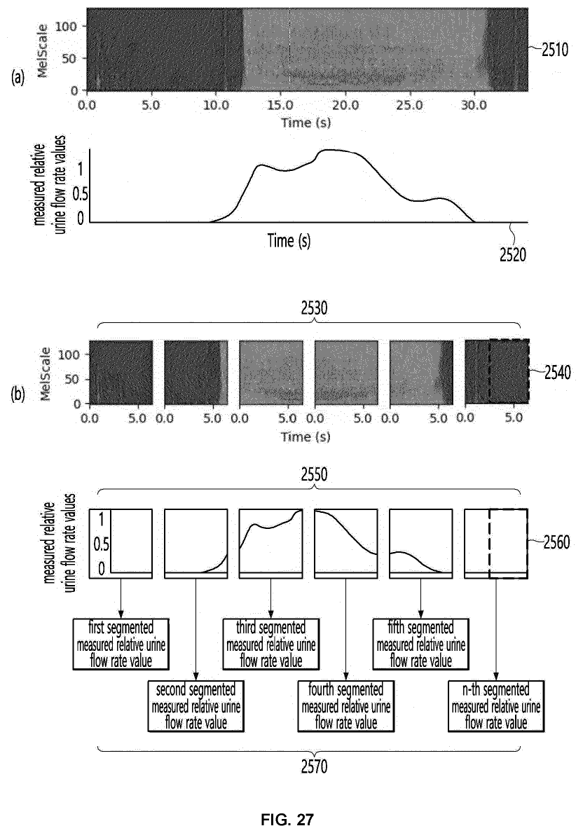

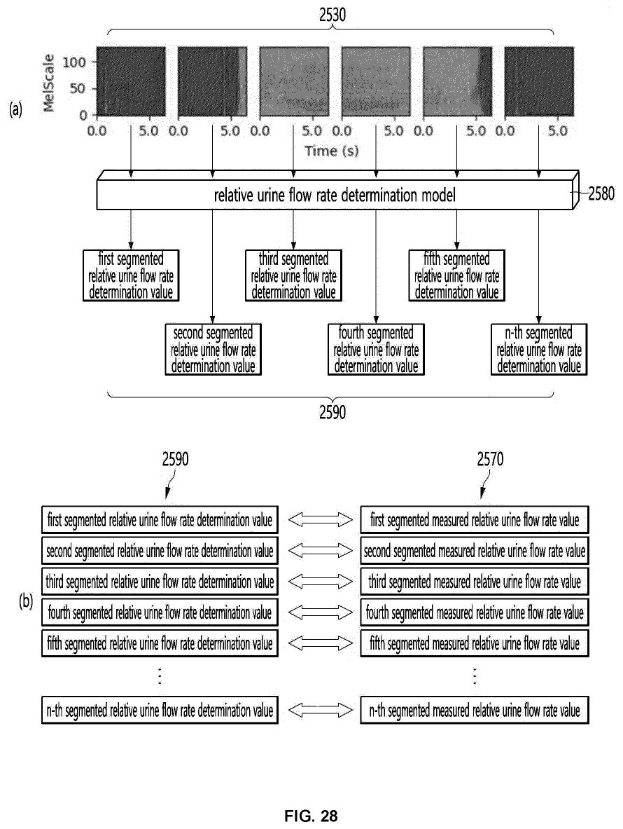

Specifically, a training process of a relative urine flow rate determination model will be described with reference to FIGS. 27 and 28.

FIGS. 27 and 28 are views describing a training process of a relative urine flow rate determination model 2580 according to the exemplary embodiment.

FIG. 27(a) shows a relative spectrogram 2510 converted from sound data recorded during a urination process and measured relative urine flow rate values 2520 corresponding to the sound data and converted from measured urine flow rate values.

FIG. 27 (b) shows segmented relative spectrograms 2530 obtained by segmenting the relative spectrogram 2510 by a predetermined time length of 6.4 seconds, and segmented measured relative urine flow rate values 2550 obtained by segmenting measured relative urine flow rate values 2520 by the predetermined time length of 6.4 seconds.

FIG. 27 shows that the spectrogram 2510 is segmented into six segmented spectrograms and the measured relative urine flow rate values 2520 is segmented into six segmented measured relative urine flow rate values, but this is according to an example, and the number of segmented spectrograms, the number of segmented measured relative urine flow rate values, and the predetermined time length are not limited thereto.

Referring to FIG. 27(b), each of time lengths of the first relative spectrogram 2510 and the measured relative urine flow rate values 2520 is not an integer multiple of the predetermined time length of 6.4 seconds, so each of time lengths of a last segmented relative spectrogram and a last segmented measured relative urine flow rate values may be shorter than the predetermined time length. In this case, as described above, padding may be performed on short time sections 2540 and 2560. As an example, zero padding for adding values of zeros may be performed. Consequently, it is possible to obtain segmented measured relative urine flow rate values 2570 having the same time lengths as each other and corresponding to respective segmented relative spectrograms 2530 having the same time lengths as each other. The segmented measured relative urine flow rate values 2570 may have consecutive values over time while in segmented time sections.

FIG. 28(a) shows that the relative urine flow rate determination model 2580 uses the segmented relative spectrograms 2530 to output segmented relative urine flow rate determination values 2590 corresponding to the segmented relative spectrograms 2530. As an example, the relative urine flow rate determination model 2580 may output a first segmented relative urine flow rate determination value corresponding to a first segmented relative spectrogram, output a second segmented relative urine flow rate determination value corresponding to a second segmented relative spectrogram, . . . , and output an n-th segmented relative urine flow rate determination value corresponding to an n-th segmented relative spectrogram.

As in FIG. 28(b), the relative urine flow rate determination model 2580 may be trained so that the respective segmented relative urine flow rate determination values 2590 correspond to the segmented measured relative urine flow rate values 2570.

The relative urine flow rate determination model 2580 may be trained through various methods such as supervised training, unsupervised training, reinforcement training, and imitation training. As an example, the relative urine flow rate determination model 2580 may be trained by comparing the segmented relative urine flow rate determination values 2590 and the segmented measured relative urine flow rate values 2570 and back-propagating an error thereof.

Meanwhile, it was described that the relative urine flow rate determination model 2580 is trained by using segmented relative spectrograms 2530 obtained by segmenting the relative spectrogram 2510 and segmented measured relative urine flow rate values 2570 obtained by segmenting the measured relative urine flow rate values 2520, but it is not limited thereto. The relative urine flow rate determination model may be configured to output relative urine flow rate determination values for the entire section by using the unsegmented relative spectrogram itself, and may also be configured to be trained by using the relative urine flow rate determination values and measured relative urine flow rate values for the entire output section.

Meanwhile, the relative urine flow rate determination model has been described as outputting the relative urine flow rate determination values by using a relative spectrogram, but it is not limited thereto, and the relative urine flow rate determination model may also be configured to output relative urine flow rate determination values by using different relative feature data or relative sound data itself other than the relative spectrogram. Since the relative feature data and relative sound data have been described above, a redundant description will be omitted.

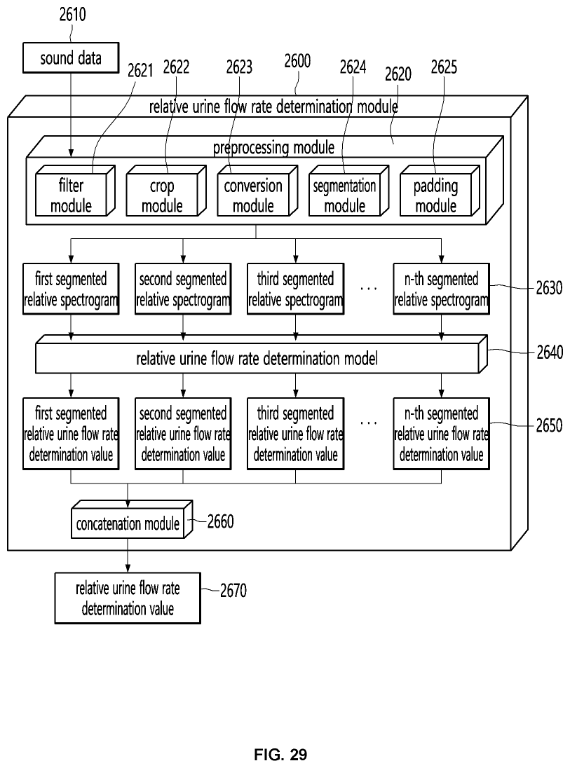

FIG. 29 is a view illustrating a configuration of a relative urine flow rate determination module 2600 according to the exemplary embodiment.

Referring to FIG. 29, the relative urine flow rate determination module 2600 may output relative urine flow rate determination values 2670 using sound data 2610. Without being limited thereto, the relative urine flow rate determination module 2600 may also be configured to output the relative urine flow rate determination value by using a spectrogram or other feature data. The relative urine flow rate determination value may refer to relative urine flow rate data, and the relative urine flow rate data may include normalized values regarding urine flow rates during a urination process.

The relative urine flow rate determination module 2600 according to one example may include a preprocessing module 2620, a relative urine flow rate determination model 2640, and a concatenation module 2660.

The preprocessing module 2620 may include a filter module 2621, a crop module 2622, a conversion module 2623, a segmentation module 2624, and/or a padding module 2625.

The filter module 2621, crop module 2622, segmentation module 2623, and padding module 2625 may operate similarly to the filter module 1621, crop module 1622, segmentation module 1624, and padding module 1625, which are included in the urine flow rate determination module 1600 described above. Since the details of the operation are described above in the part describing the urine flow rate determination module, a redundant description will be omitted.

In a case where the relative urine flow rate determination model 2640, which will be described below, is a model trained to output relative urine flow rate determination values by using a relative spectrogram, the conversion module 2623 may convert sound data 2610 into a spectrogram, and may convert the spectrogram into a relative spectrogram by dividing all values by a maximum value among the values of the converted spectrogram. Without being limited thereto, the conversion module 2623 may also convert the spectrogram into the relative spectrogram through a process of normalizing the values of the spectrogram.

Meanwhile, in a case where the relative urine flow rate determination model 2640 is trained to output relative urine flow rate determination values by using other relative feature data, the conversion module 2623 may also convert the sound data 2610 into other feature data, and convert the feature data into relative feature data through a process of normalizing the values of the converted feature data. Since the types of feature data have been described above, a redundant description will be omitted.

Meanwhile, in a case where the relative urine flow rate determination model 2640 is trained to output relative urine flow rate determination values by using relative sound data, the conversion module 2623 may also convert the sound data into the relative sound data through a process of normalizing the values of the sound data 2610.

The relative urine flow rate determination model 2640 is a model trained to receive an input of data of a urination process and output data including normalized values about urine flow rates during a urination process. As an example, the relative urine flow rate determination model 2640 may be a model trained to output relative urine flow rate determination values by using a relative spectrogram, and may be a model trained in the training method of the relative urine flow rate determination model described above. Without being limited thereto, the relative urine flow rate determination module 2640 may also be model trained to output relative urine flow rate determination values by using relative sound data or other relative feature data.

Referring to FIG. 29, the relative urine flow rate determination model 2640 may be configured to output first to n-th segmented relative urine flow rate determination values 2650 corresponding to respective segmented relative spectrograms by using the first to n-th segmented relative spectrograms 2630 segmented by the segmentation module 2624.

The concatenation module 2660 may be configured to output the relative urine flow rate determination values 2670 corresponding to the sound data 2610 by concatenating the first to n-th segmented relative urine flow rate determination values 2650 over time. The concatenation module 2660 may operate similarly to the concatenation module 1660 included in the urine flow rate determination module 1600, and since the details of the operation are described above in the part describing the urine flow rate determination module, a redundant description will be omitted.

As described above, the relative urine flow rate determination module 2600 segments the relative spectrogram by the predetermined length, obtains the segmented relative urine flow rate determination values by using the segmented relative spectrograms, and obtains all entire relative urine flow rate determination values by concatenating the segmented relative urine flow rate determination values. Even though sound data of various lengths is input, the relative urine flow rate determination module may determine the relative urine flow rate by using the relative spectrogram of a predetermined length, so there is an effect of reducing the influence of the total length of the sound data on the accuracy of a relative urine flow rate determination result.

The relative urine flow rate determination module 2600 normalizes a spectrogram to convert it into a relative spectrogram, and obtains a relative urine flow rate determination value by using the relative spectrogram, so there is an effect of reducing the influence of an absolute magnitude of sound obtained during a urination process on the determination value.

Meanwhile, the relative urine flow rate determination module 2600 has been described as receiving an input of the sound data 2610 and outputting the relative urine flow rate determination values 2670, but it is not limited thereto. The relative urine flow rate determination module may also be configured to receive an input of a spectrogram or other feature data itself and output a relative urine flow rate determination value. In this case, the conversion module 1623 may omit the operation of converting the sound data into the spectrogram or other feature data.

Meanwhile, the relative urine flow rate determination module may also be configured to receive an input of relative sound data, a relative spectrogram, or other relative feature data itself and output a relative urine flow rate determination value. In this case, the conversion module 2623 may not be included in the preprocessing module 2620.

Meanwhile, the relative urine flow rate determination module 2600 is not required to include all the filter module 2621, crop module 2622, conversion module 2623, segmentation module 2624, and padding module 2625. As an example, the relative urine flow rate determination module 2600 may include merely some of the filter module 2621, crop module 2622, conversion module 2623, segmentation module 2624, and padding module 2625, or may also not include the preprocessing module 2620 itself. In a case when the relative urine flow rate determination module 2600 does not include the segmentation module 2624, the relative urine flow rate determination module 2600 may also not include the concatenation module 2660. In a case where the relative urine flow rate determination module 2600 does not include the preprocessing module 2620, the relative urine flow rate determination module 2600 may also be configured to output a relative urine flow rate determination value by using the relative sound data, relative spectrogram, or other relative feature data itself.

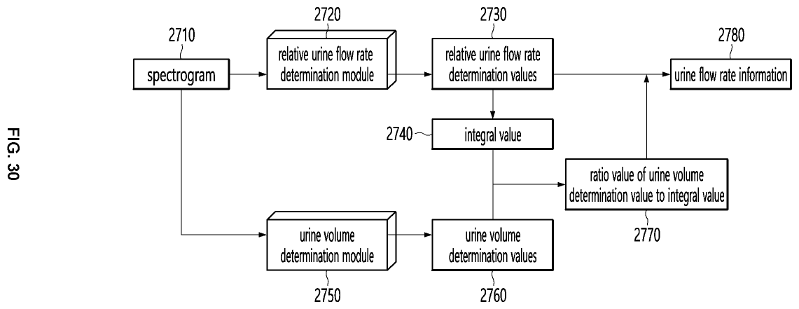

In order to obtain highly accurate urine flow rate information, the relative urine flow rate determination module, urine volume determination module, and/or urination presence/absence determination module may also be used together. Specific details will be described with reference to FIGS. 30 to 32.

FIG. 30 is a view illustrating a method of obtaining urine flow rate information according to the exemplary embodiment

Referring to FIG. 30, the sound analysis system may obtain urine flow rate information 2780 by using a relative urine flow rate determination module 2720 and a urine volume determination module 2750. Specifically, the sound analysis system may obtain the urine flow rate information 2780 by applying urine volume determination values 2760 output by the urine volume determination module 2750 to relative urine flow rate determination values 2730 output by the relative urine flow rate determination module 2720.

More specifically, the sound analysis system may obtain the relative urine flow rate determination values 2730 by using the spectrogram 2710 and the relative urine flow rate determination module 2720. In addition, the sound analysis system may obtain urine volume determination values 2760 by using the spectrogram 2710 and the urine volume determination module 2750.

The spectrogram may be obtained by using sound data recorded during a urination process. Since the content related to using the spectrogram to output the relative urine flow rate determination values and the urine volume determination values by the relative urine flow rate determination module and the urine volume determination module has been described above, a redundant description will be omitted.

FIG. 30 shows that the relative urine flow rate determination module 2720 is configured to output the relative urine flow rate determination values 2730 by using the spectrogram 2710, and the urine volume determination module 2740 is configured to output the urine volume determination values 2760 by using the spectrogram 2710, but it is not limited thereto. The relative urine flow rate determination module 2720 and/or the urine volume determination module 2740 may also be configured to output determination values by using sound data or other feature data, and since the specific details thereof have been described above, a redundant description will be omitted.

The sound analysis system may obtain an integral value 2740 by integrating the relative urine flow rate determination values 2730 over time. A urine flow rate refers to a urine volume per unit time, so in a case where the relative urine flow rate determination values 2730 are integrated over time, a relative urine volume may be obtained as the integral value 2740.

The sound analysis system may calculate a ratio value 2770 of each urine volume determination value 2760 to the obtained integral value 2740. A difference between the relative urine volume value obtained from the relative urine flow rate determination module 2720 and the urine volume determination values 2760 obtained from urine volume determination module 2750 may be reflected in the ratio value 2770.

The sound analysis system may obtain urine flow rate information 2780 by applying the calculated ratio value 2770 to each relative urine flow rate determination value 2730.

Applying the ratio value 2770 to the relative urine flow rate determination value 2730 may refer to multiplying each of the relative urine flow rate determination values 2730 over time by the ratio value 2770. Without being limited thereto, the applying of the ratio value 2770 to each relative urine flow rate determination value 2730 may refer to adjusting each of the relative urine flow rate determination values 2730 by using the ratio value 2770.

In a case of the relative urine flow rate determination values 2730 output by the relative urine flow rate determination module 2720, the influence of an absolute magnitude of sound may be small, so the accuracy of a urine flow rate waveform of the relative urine flow rate determination values 2730 may be higher than that of a urine flow rate waveform output by the urine flow rate determination module. Accordingly, more accurate urine flow rate information 2780 may be obtained by applying the ratio value 2770 to the highly accurate urine flow rate waveform.

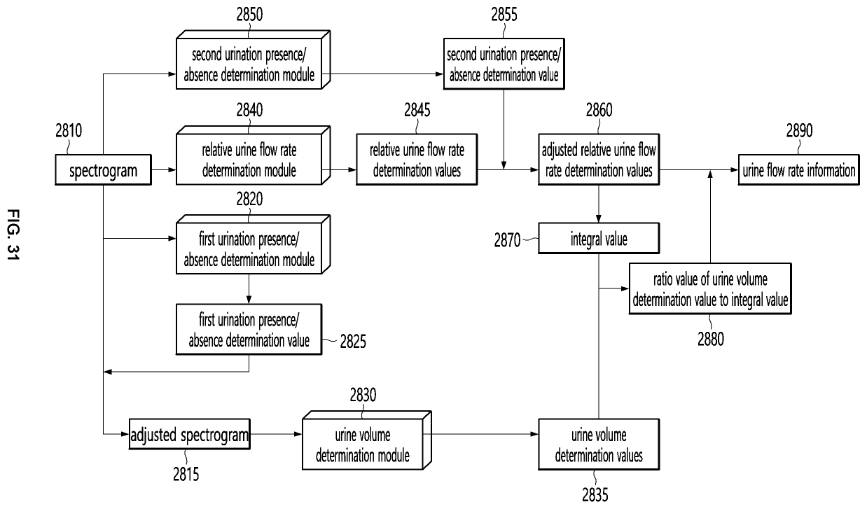

Meanwhile, the sound analysis system may also obtain urine flow rate information by using all the relative urine flow rate determination module, urine volume determination module, and urination presence/absence determination module. Specific details will be described with reference to FIG. 31.

FIG. 31 is a view illustrating a method of obtaining urine flow rate information according to the exemplary embodiment.

Referring to FIG. 31, the sound analysis system may obtain urine volume determination values 2835 by using a first urination presence/absence determination module 2820 and a urine volume determination module 2830, obtain adjusted relative urine flow rate determination values 2860 by using a second urination presence/absence determination module 2850 and a relative urine flow rate determination module 2840, and obtain urine flow rate information 2890 by using the adjusted relative urine flow rate determination values 2860 and urine volume determination values 2835, which are obtained.

The first urination presence/absence determination module 2820 and the second urination presence/absence determination module 2850 may be configured with the same urination presence/absence determination modules, but it is not limited thereto. The first urination presence/absence determination module 2820 and the second urination presence/absence determination module 2850 may also be configured with urination presence/absence determination modules different from each other. As an example, the first urination presence/absence determination module 2820 may be a model trained to output a probability value for urination presence/absence, and the second urination presence/absence determination module 2850 may be a model trained to output a classification value for classifying the urination presence/absence, and vice versa.

The sound analysis system may obtain a first urination presence/absence determination value 2825 by using a spectrogram 2810 and the first urination presence/absence determination module 2820. The sound analysis system may obtain an adjusted spectrogram 2815 by applying the obtained first urination presence/absence determination value 2825 to the spectrogram 2810. In addition, the sound analysis system may obtain urine volume determination values 2835 by using the adjusted spectrogram 2815 and the urine volume determination module 2830.

The spectrogram may be obtained by using sound data recorded during a urination process. Since the content related to the sound analysis system obtaining the urine volume determination values by using the urination presence/absence determination module and the urine volume determination module together has been described in detail in FIGS. 12 and 13, a redundant description will be omitted.

In FIG. 31, the first urination presence/absence determination module 2820 is configured to output the first urination presence/absence determination value 2825 by using the spectrogram 2810, and the urination amount determination module 2830 is also configured to output the urine volume determination values 2835 by using the spectrogram 2815, but it is not limited thereto. The first urination presence/absence determination module 2820 and/or the urine volume determination module 2830 may also be configured to output determination values by using sound data or other feature data, and since the specific details thereof have been described above, a redundant description will be omitted.

The sound analysis system may obtain relative urine flow rate determination values 2845 by using the spectrogram 2810 and the relative urine flow rate determination module 2840. In addition, the sound analysis system may obtain a second urination presence/absence determination value 2855 by using the spectrogram 2810 and a second urination presence/absence determination module 2850. The second urination presence/absence determination value 2855 may be the same as the first urination presence/absence determination value 2825, but may also have a different value.

The spectrogram may be obtained by using sound data recorded during a urination process. Since the content related to the sound analysis system obtaining the relative urine flow rate determination values by using the spectrogram and the relative urine flow rate determination module have been described above, a redundant description will be omitted.

A method of obtaining, by the sound analysis system, the adjusted relative urine flow rate determination values 2860 by using the relative urine flow rate determination module 2840 and the urination presence/absence determination module 2850 together may be performed similarly to a method of obtaining, by the sound analysis system, the adjusted urine flow rate determination values by using the urine flow rate determination module and the urination presence/absence determination module, and since the content related to the method of obtaining, by the sound analysis system, the adjusted urine flow rate determination values by using the urine flow rate determination module and the urination presence/absence determination module together is described above in FIGS. 19 and 20, a redundant description will be omitted.

In FIG. 31, the relative urine flow rate determination module 2840 is configured to output the relative urine flow rate determination values 2845 by using the spectrogram 2810, and the second urination presence/absence determination module 2850 is also configured to output the second urination presence/absence determination value 2855 by using the spectrogram 2810, but it is not limited thereto. The relative urine flow rate determination module 2840 and/or the second urination presence/absence determination module 2850 may also be configured to output determination values by using sound data or other feature data, and since the specific details thereof have been described above, a redundant description will be omitted.

The sound analysis system may obtain urine flow rate information 2890 by using the adjusted relative urine flow rate determination values 2860 and urine volume determination values 2835, which are obtained. Specifically, the sound analysis system may obtain an integral value 2870 by integrating the adjusted relative urine flow rate determination values 2860 over time, and calculate a ratio value 2880 of each urine volume determination value 2835 to the integral value 2870. Since the content related to the integral value and ratio value have been described above in FIG. 21, a redundant description will be omitted.

The sound analysis system may obtain much more accurate urine flow rate information 2890 by applying the calculated ratio value 2880 to the adjusted relative urine flow rate determination values 2860. The applying of the ratio value 2880 to the adjusted relative urine flow rate determination values 2860 may refer to multiplying each of the adjusted relative urine flow rate determination values 2860 over time by the ratio value 2880. Not being limited to this, the applying of the ratio value 2880 to the adjusted relative urine flow rate determination values 2860 may include adjusting the adjusted relative urine flow rate determination values 2860 by using the ratio value 2880.

In a case of the relative urine flow rate determination values 2845 output by the relative urine flow rate determination module 2840, the influence of an absolute magnitude of sound may be small, so the accuracy of a urine flow rate waveform of the relative urine flow rate determination values 2845 may be high. Accordingly, highly accurate urine flow rate information 2890 may be obtained by applying the ratio value 2880 to a urine flow rate waveform having high accuracy.

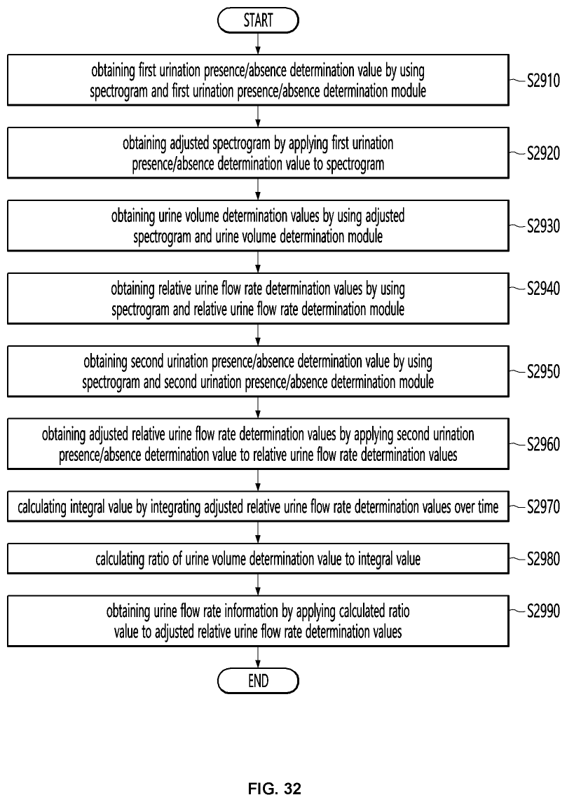

FIG. 32 is a flowchart illustrating a method of obtaining urine flow rate information according to the exemplary embodiment.

Referring to FIG. 32, in step S2910, the sound analysis system may obtain a first urination presence/absence determination value by using a spectrogram and a first urination presence/absence determination module. The spectrogram may have been obtained by using sound data recorded during a urination process. Since the content related to using the spectrogram to output the urination presence/absence determination values by the urination presence/absence determination module has been described above, a redundant description will be omitted.

Meanwhile, in a case where the urination presence/absence determination module is trained to output urination presence/absence determination values by using sound data, the sound analysis system may also obtain urination presence/absence determination values by using the sound data and the urination presence/absence determination module.

In step S2920, the sound analysis system may obtain an adjusted spectrogram by applying the first urination presence/absence determination value to the spectrogram. Since the content related to obtaining the adjusted spectrogram have been described above, a redundant description will be omitted.

In step S2930, the sound analysis system may obtain urine volume determination values by using the adjusted spectrogram and a urine volume determination module. Since the content related to the urine volume determination module using the spectrogram to output the urine volume determination values has been described above, a redundant description will be omitted.

In step S2940, the sound analysis system may obtain relative urine flow rate determination values by using the spectrogram and a relative urine flow rate determination module. Since the content related to using the spectrogram to output the relative urine flow rate determination values by the relative urine flow rate determination module has been described above, a redundant description will be omitted.

Meanwhile, in a case where the relative urine flow rate determination module is trained to output relative urine flow rate determination values by using sound data, the sound analysis system may also obtain relative urine flow rate determination values by using the sound data and the relative urine flow rate determination module.

In step S2950, the sound analysis system may obtain a second urination presence/absence determination value by using the spectrogram and a second urination presence/absence determination module. The second urination presence/absence determination module may be the same urination presence/absence determination module as the first urination presence/absence determination module, but it is not limited thereto. The first urination presence/absence determination module and the second urination presence/absence determination module may also be configured with urination presence/absence determination modules different from each other, and since the specific contents have been described above, a redundant description will be omitted.

In step S2960, the sound analysis system may obtain adjusted relative urine flow rate determination values by applying the second urination presence/absence determination value to the relative urine flow rate determination values. Since the content related to obtaining the adjusted relative urine flow rate determination values by using the urination presence/absence determination values and the relative urine flow rate determination values have been described above, a redundant description will be omitted.

In step S2970, the sound analysis system may calculate an integral value by integrating the adjusted relative urine flow rate determination values over time. In step S2980, the sound analysis system may calculate a ratio of a urine volume determination value to the integral value. Since the content related to the integral value and ratio value have been described above, a redundant description will be omitted.

In step S2990, the sound analysis system may obtain urine flow rate information by applying the calculated ratio value to the adjusted relative urine flow rate determination values. Since the content related to the applying of the ratio value to the adjusted relative urine flow rate determination values has been described in detail, a redundant description will be omitted.

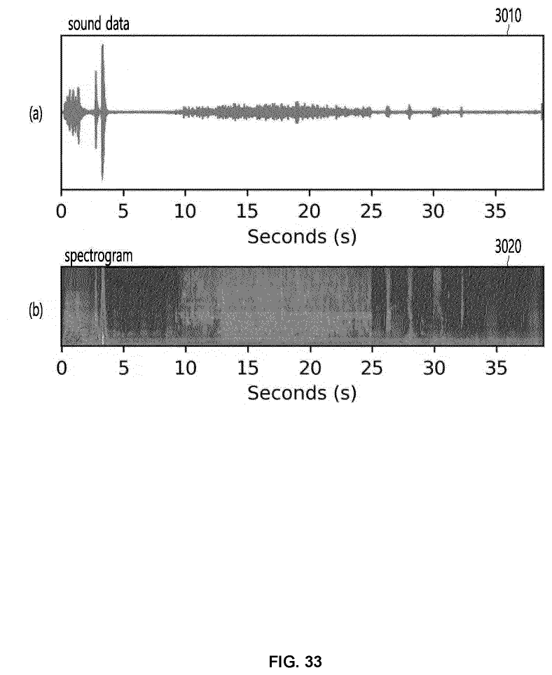

FIGS. 33, 34, 35, 36, 37, and 38 are views illustrating urination information obtained according to the exemplary embodiment.

Referring to FIG. 33, FIG. 33(a) is a view illustrating sound data 3010 obtained by recording sound during a urination process according to an example, and FIG. 33(b) is a view illustrating a spectrogram 3020 converted from the sound data in FIG. 33(a) according to the example.

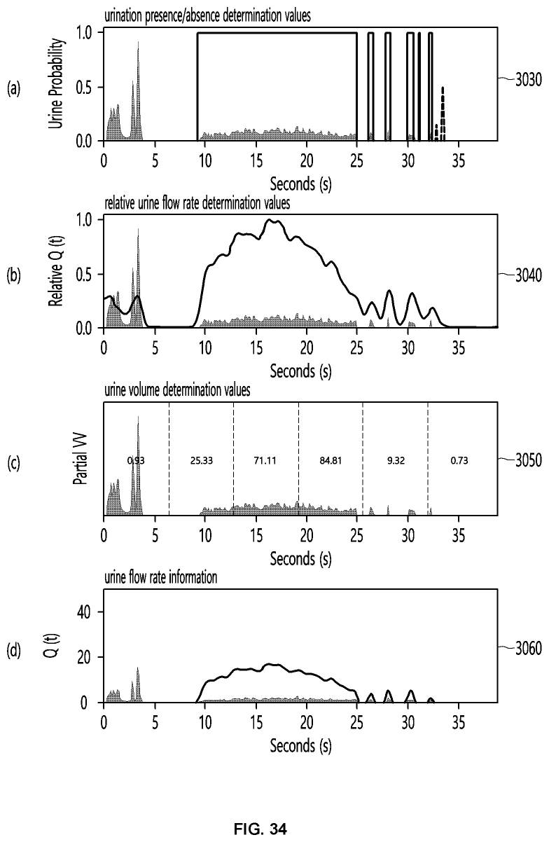

Referring to FIG. 34, FIG. 34(a) is a view illustrating urination presence/absence determination values 3030 obtained by using a urination presence/absence determination module and the spectrogram 3020 according to the exemplary embodiment.

FIG. 34(b) is a view illustrating relative urine flow rate determination values 3040 obtained by using the relative urine flow rate determination module and the spectrogram 3020 according to the exemplary embodiment.

FIG. 34(c) is a view illustrating urine volume determination values 3050 obtained by using a urine volume determination module and the spectrogram 3020 according to the exemplary embodiment. A number written for each section shown in FIG. 34(c) represents a urine volume determination value 3050 for each predetermined time section over time.

FIG. 34(d) is a view illustrating urine flow rate information 3060 obtained by using the urination presence/absence determination values 3030, the relative urine flow rate determination values 3040, and the urine volume determination values 3050 according to the exemplary embodiment.

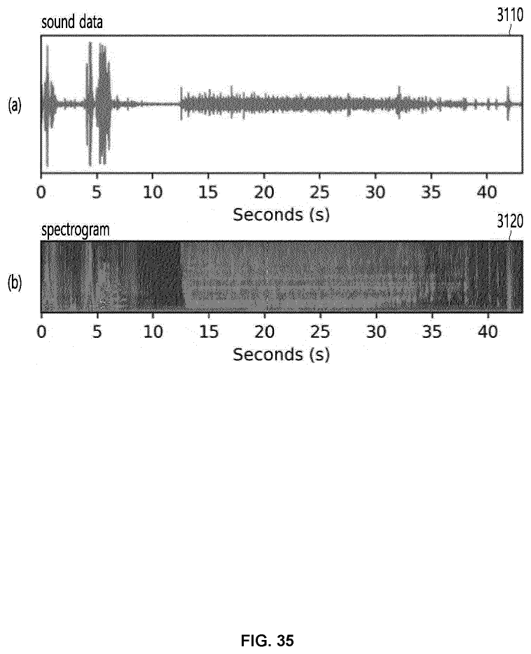

FIG. 35(a) is a view illustrating sound data 3110 obtained by recording sound during a urination process according to an example, and FIG. 35(b) is a view illustrating a spectrogram 3120 converted from the sound data in FIG. 35(a) according to the example.

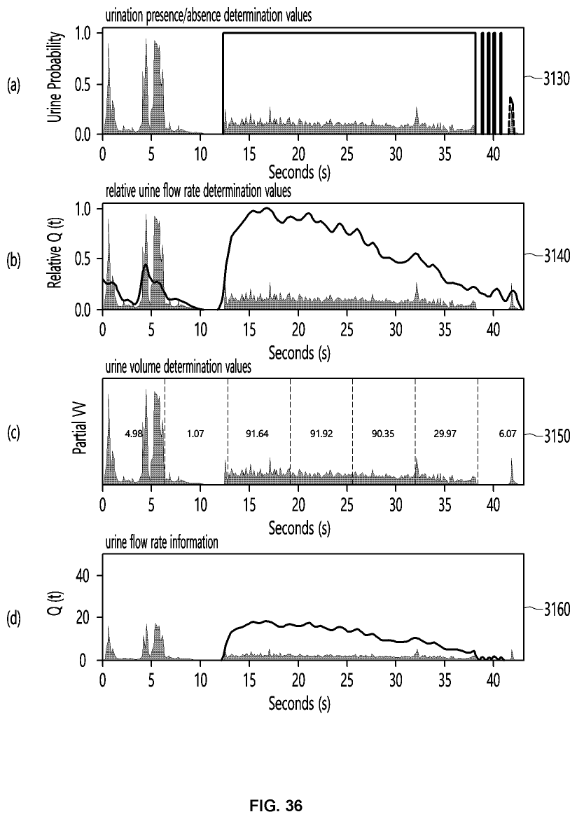

Referring to FIG. 36, FIG. 36(a) is a view illustrating urination presence/absence determination values 3130 obtained by using the urination presence/absence determination module and the spectrogram 3120 according to the exemplary embodiment.

FIG. 36(b) is a view illustrating relative urine flow rate determination values 3140 obtained by using the relative urine flow rate determination module and the spectrogram 3120 according to the exemplary embodiment.

FIG. 36(c) is a view illustrating urine volume determination values 3150 obtained by using the urination amount determination module and the spectrogram 3120 according to the exemplary embodiment. A number written for each section shown in FIG. 36(c) represents a urine volume determination value 3150 for each predetermined time section over time.

FIG. 36(d) is a view illustrating urine flow rate information 3160 obtained by using the urination presence/absence determination values 3130, the relative urine flow rate determination values 3140, and the urine volume determination values 3150 according to the exemplary embodiment.

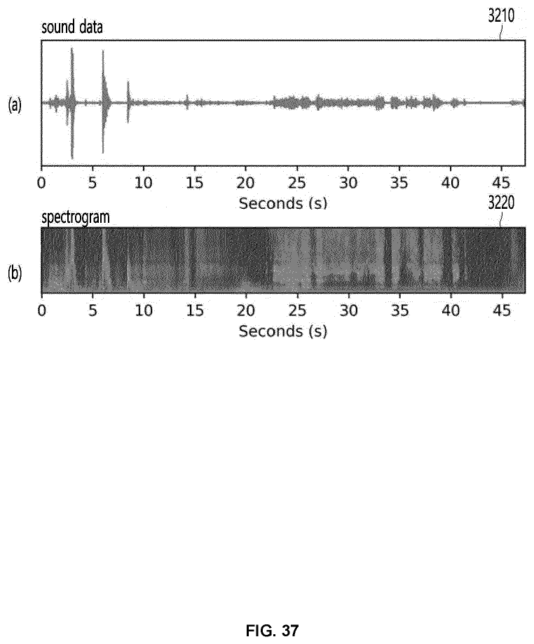

Referring to FIG. 37, FIG. 37(a) is a view illustrating sound data 3210 obtained by recording sound during a urination process according to an example, and FIG. 37(b) is a view illustrating a spectrogram 3220 converted from the sound data in FIG. 37(a) according to the example.

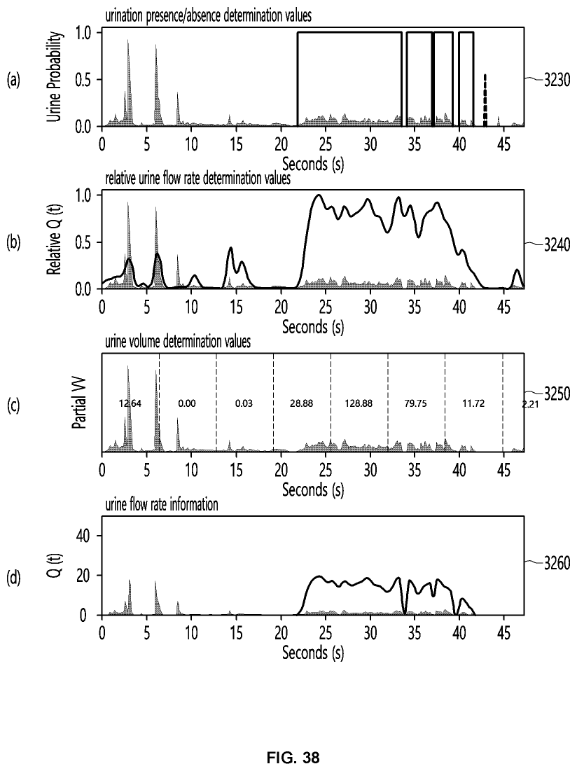

Referring to FIG. 38, FIG. 38(a) is a view illustrating urination presence/absence determination values 3230 obtained by using the urination presence/absence determination module and the spectrogram 3220 according to the exemplary embodiment.

FIG. 38(b) is a view illustrating relative urine flow rate determination values 3240 obtained by using a relative urine flow rate determination module and the spectrogram 3220 according to the exemplary embodiment.

FIG. 38(c) is a view illustrating urine volume determination values 3250 obtained by using the urine volume determination module and the spectrogram 3220 according to the exemplary embodiment. A number written for each section shown in FIG. 38(c) represents a urine volume determination value 3250 for each predetermined time section over time.

FIG. 38(d) is a view illustrating urine flow rate information 3260 obtained by using the urination presence/absence determination values 3230, the relative urine flow rate determination values 3240, and the urine volume determination values 3250 according to the exemplary embodiment.

The methods according to the exemplary embodiment may be implemented in the form of program instructions that may be executed through various computer means, and may be recorded in a computer-readable media. The computer-readable media may include program instructions, data files, data structures, and the like alone or in combination. The program instructions recorded on the media may be designed and configured specifically for the exemplary embodiments or may be publicly known and available to those skilled in the art regarding computer software. Examples of the computer-readable recording media include a magnetic medium such as a hard disk, a floppy disk, and a magnetic tape, an optical medium such as a CD-ROM, a DVD, a magneto-optical medium such as a floptical disk, and a hardware device specially configured to store and perform program instructions, the hardware device including a read-only memory (ROM), a random access memory (RAM), a flash memory, etc. Examples of the computer instructions include not only machine language code generated by a compiler, but also high-level language code executable by a computer using an interpreter or the like. The hardware device described above may be configured to operate by one or more software modules to perform the operations of the exemplary embodiments, and vice versa.

The present disclosure described above is capable of various substitutions, modifications, and changes without departing from a scope of the technical idea of the present disclosure for those skilled in the art to which the present disclosure pertains, so the present disclosure is not limited by the above-described exemplary embodiments and attached drawings. In addition, the exemplary embodiments described in the present document are not intended to be limited in application, but all or part of each of the exemplary embodiments may also be selectively combined and configured so that various modifications may be made. Furthermore, the steps constituting each exemplary embodiment may be used individually or in combination with the steps constituting other exemplary embodiments.

1. A method of obtaining urination information, comprising:
obtaining one or more first feature data by using first sound data, wherein the first sound data reflect a sound of a urination process;
obtaining a urine volume determination value by using the one or more first feature data and a pre-trained urine volume determination model, wherein the urine volume determination model is trained with a urine volume training data set, wherein the urine volume training data set comprises one or more second feature data generated based on second sound data recorded during a urination process and a value related to a urine volume corresponding to the second sound data;
obtaining a urine flow rate determination value by using the one or more first feature data and a pre-trained urine flow rate determination model, wherein the urine flow rate determination model is trained with a urine flow rate training data set, wherein the urine flow rate training data set comprises one or more third feature data generated based on third sound data recorded during a urination process and a value related to a urine flow rate corresponding to the third sound data; and
obtaining a urine flow rate information by reflecting a ratio of an estimated urine volume calculated based on the urine flow rate determination value and the urine volume determination value to the urine flow rate determination value.

2. The method of claim 1,
wherein the estimated urine volume is calculated by integrating the urine flow rate determination value over time.

3. The method of claim 1,
wherein the method comprises:
obtaining a urination presence/absence determination value by using the first feature data and a pre-trained urination presence/absence determination model, wherein the urination presence/absence determination model is trained with a urination presence/absence training data set, wherein the urination presence/absence training data set comprises one or more fourth feature data generated based on fourth sound data recorded during a urination process and a value related to a urination presence/absence corresponding to the fourth sound data,
wherein the obtaining the urine volume determination value comprises:
obtaining one or more adjusted first feature data by reflecting the urination presence/absence determination value to the one or more first feature data; and
obtaining the urine volume determination value by using the one or more adjusted first feature data and the urine volume determination model.

4. The method of claim 3,
wherein the method comprises:
obtaining a urination presence/absence classification value by using the urination presence/absence determination value, wherein the urination presence/absence classification value is either a urination section indication value or a non-urination section indication value, determined according to the urination presence/absence determination value; and
obtaining an adjusted urine flow rate determination value by reflecting the urination presence/absence classification value to the urine flow rate determination value;
wherein the estimated urine volume is calculated by integrating the adjusted urine flow rate determination value over time.

5. The method of claim 1,
wherein the one or more first feature data is generated by transforming the first sound data into a spectrogram and segmenting the spectrogram into a plurality of segmented spectrograms having a preset time length.

6. The method of claim 5,
wherein the obtaining a urine volume determination value comprises:
obtaining a plurality of segmented urine volume determination value for each of the plurality of segmented spectrograms by inputting each of the plurality of segmented spectrograms into the urine volume determination model; and
obtaining the urine volume determination value by adding the plurality of segmented urine volume determination value.

7. The method of claim 5,
wherein the method comprises:
when a time length of a last segmented spectrogram among the plurality of segmented spectrogram is shorter than the preset time length, padding on the last segmented spectrogram.

8. A method of obtaining urination information, comprising:
obtaining one or more first feature data and one or more first relative feature data by using first sound data, wherein the first sound data reflect a sound of a urination process, and the first relative feature data comprise normalized value;
obtaining a urine volume determination value by using the one or more first feature data and a pre-trained urine volume determination model, wherein the urine volume determination model is trained with a urine volume training data set, wherein the urine volume training data set comprises one or more second feature data generated based on second sound data recorded during a urination process and a value related to a urine volume corresponding to the second sound data;
obtaining a relative urine flow rate determination value by using the one or more first relative feature data and pre-trained relative urine flow rate determination model, wherein the relative urine flow rate determination model is trained with a relative urine flow rate training data set, wherein the relative urine flow rate training data set comprises one or more second relative feature data generated based on third sound data recorded during a urination process and a value related to a relative urine flow rate corresponding to the third sound data; and
obtaining a urine flow rate information by reflecting a ratio of an integral value calculated based on the relative urine flow rate determination value and the urine volume determination value to the relative urine flow rate determination value.

9. The method of claim 8,
wherein the integral value is calculated by integrating the relative urine flow rate determination value over time.

10. The method of claim 8,
wherein the method comprises:
obtaining a urination presence/absence determination value by using the one or more first feature data and pre-trained urination presence/absence determination model, wherein the urination presence/absence determination model is trained with a urination presence/absence training data set, wherein the urination presence/absence training data set comprises one or more third feature data generated based on fourth sound data recorded during a urination process and a value related to a urination presence/absence corresponding to the fourth sound data;
obtaining a urination presence/absence classification value by using the urination presence/absence determination value, wherein the urination presence/absence classification value is either a urination section indication value or a non-urination section indication value, determined according to the urination presence/absence determination value; and
obtaining an adjusted urine flow rate determination value by reflecting the urination presence/absence classification value to the urine flow rate determination value;
wherein the obtaining the urine volume determination value comprises:
obtaining one or more adjusted first feature data by reflecting the urination presence/absence determination value to the one or more first feature data; and
obtaining the urine volume determination value by using the one or more adjusted first feature data and the urine volume determination model,
wherein the integral value is calculated by integrating the adjusted urine flow rate determination value over time.

11. The method of claim 8,
wherein the one or more first feature data is generated by transforming the first sound data into a spectrogram and segmenting the spectrogram into a plurality of segmented spectrograms having a preset time length,
wherein the obtaining the urine volume determination value comprises:
obtaining plurality of segmented urine volume determination value for each of the plurality of segmented spectrograms by inputting each of the plurality of segmented spectrograms into the urine volume determination model; and
obtaining the urine volume determination value by adding the plurality of segmented urine volume determination value.

12. A computer-readable non-transitory recording medium storing a method of obtaining urination information with high accuracy,
wherein the method of obtaining urination information is a method of obtaining urination information according to claim 1.

13. A sound analysis system, comprising:
a memory storing first sound data, pre-trained urine volume determination model and pre-trained urine flow rate determination model, wherein the first sound data reflect a sound of a urination process, the urine volume determination model is trained with a urine volume training data set comprising one or more first feature data generated based on second sound data recorded during a urination process and a value related to a urine volume corresponding to the second sound data, and the urine flow rate determination model is trained with a urine flow rate training data set comprising one or more second feature data generated based on third sound data recorded during a urination process and a value related to urine flow rate corresponding to the third sound data; and
at least one processor,
wherein the processor:
obtain one or more third feature data by using the first sound data, obtain a urine volume determination value by using the one or more third feature data and the urine volume determination model, obtain a urine flow rate determination value by using the one or more third feature data and the urine flow rate determination model, and obtain a urine flow rate information by reflecting a ratio of an estimated urine volume calculated based on the urine flow rate determination value and the urine volume determination value to the urine flow rate determination value.

14. A sound analysis system, comprising:
a memory storing first sound data, pre-trained urine volume determination model and pre-trained relative urine flow rate determination model, wherein the first sound data reflect a sound of a urination process, the urine volume determination model is trained with a urine volume training data set comprising one or more first feature data generated based on second sound data recorded during a urination process and a value related to a urine volume corresponding to the second sound data, and the relative urine flow rate determination model is trained with a relative urine flow rate training data set comprising one or more first relative feature data generated based on third sound data recorded during a urination process and a value related to relative urine flow rate corresponding to the third sound data; and
at least one processor,
wherein the processor:
obtain one or more second feature data and one or more second relative feature data by using the first sound data, obtain a urine volume determination value by using the one or more second feature data and the urine volume determination model, obtain a relative urine flow rate determination value by using the one or more second relative feature data and the relative urine flow rate determination model, and obtain a urine flow rate information by reflecting a ratio of an integral value calculated based on the relative urine flow rate determination value and the urine volume determination value to the relative urine flow rate determination value.
